(19) United States
(12) Patent Application Publication
(10) Pub. No.: US 2019/0037899 A1
(43) Pub. Date: Feb. 7, 2019

(54) Intramolecular Trans-Sialidase

(71) Applicant: THE INSTITUTE OF FOOD RESEARCH, Norwich (GB)

(72) Inventors: Nathalie Juge, Norwich (GB); Marie Maguelonne Monestier, Norwich (GB); Robert Andrew Field, Norwich (GB)

(21) Appl. No.: 16/075,485

(22) PCT Filed: Feb. 6, 2017
(86) PCT No.: PCT/GB2017/050291

§ 371 Date: Aug. 3, 2018

(30) Foreign Application Priority Data
Feb. 5, 2016 (GB) 1602105.7

(51) Int. Cl. A23L 33/10; A23L 29/00; A61K 38/47; A61P 31/16; A61P 31/04; C12N 9/26; C12R 1/225; C12N 15/74

(52) U.S. Cl. CPC A23L 33/10; A23L 29/06; A61K 38/47; A61P 31/16; C12Y 302/01018; C12N 9/2411; C12R 1/225; C12N 15/74; A61P 31/04

(57) ABSTRACT

The present invention relates to an intramolecular trans-sialidase (IT-sialidase) enzyme from Ruminococcus gnavus and fragments thereof. It also relates to methods of production of the IT-sialidase or its fragments, in addition to uses of the IT-sialidase, its fragments, or compositions comprising the IT-sialidase. One of the uses of the IT-sialidase or its fragments encompassed by the present invention is a method for production of 2,7-anhydro sialic acid derivatives. Further uses of the IT-sialidase enzyme include the therapeutic and prophylactic treatment of infections and respiratory conditions.

### TECHNICAL FIELD OF THE INVENTION

The disclosure herein relates to the identification of an intramolecular trans-sialidase (IT-sialidase) and to intramolecular trans-sialidase (IT-sialidase)-expressing bacteria. The present invention further relates to compositions and uses of said intramolecular trans-sialidase enzyme and the enzyme expressing bacteria in medicine, in particular for the therapeutic and prophylactic treatment of infections and respiratory conditions.

### BACKGROUND OF THE INVENTION

In humans, microbial infection typically starts with bacteria interacting with the mucosal surfaces which are more exposed and prone to infection (such as those in the gut and the respiratory tract). The mucins are components of mucus gel layers at mucosal surfaces throughout the body that play roles in protection as part of the defensive barrier on an organ and tissue specific basis. Mucins provide binding sites, and a source of nutrients for microorganisms, and their dysregulation is associated with numerous diseases such as cancer, infections and inflammatory bowel diseases. Therefore, strategies targeting mucin glycans from the human intestinal and respiratory tract are needed for the management of the human mucosal defensive barrier.

Airway mucus hypersecretion and stasis of tenacious mucus is a characteristic feature in cystic fibrosis (CF), asthma, and a number of other severe respiratory conditions like COPD (chronic obstructive pulmonary disease). There is currently a great unmet medical need for new, highly potent mucolytic agents that can speed up the breakdown and clearance of pathological mucus. The pathologies of these diseases are exacerbated by a shift in the composition of mucosal microbes associated with, or consequent to, changes in the mucus layer. Mucus hypersecretion and stasis accelerates the decrease in lung function, enables inhaled pathogens to multiply and to cause lung infections, and increases patients' risk of death. Consequently, in the treatment of these patients the removal of the tenacious airway secretions is a major goal. Compounds like rhDNase-I and N-acetylcysteine derivatives that can lower mucus viscosity and elasticity, and thus increase the clearance of the mucus out of the lungs, are available for clinical use. In clinical trials, rhDNase-I has shown to be effective in enhancing lung function and decreasing acute exacerbations in CF. The individual response is, however, highly variable and an important group of CF patient does not benefit from rhDNase-I. Also, there is no evidence that CF or COPD patients benefit from N acetylcysteine and, consequently, this drug and its derivatives are not included in guidelines for the management of asthma and COPD patients. A disadvantage of N-acetylcysteine-based drugs lies in the fact that they are consumed by the reaction with mucins and therefore have to be present in equivalent amounts to the molecules they are targeting. The latter is difficult to achieve in vivo. A key component of the mucosal surface interaction is sialic acid (Neu5Ac), a sugar residue commonly found in mucus secretions in a terminal location of intestinal and respiratory mucins. This sugar provides a major point of attachment for pathogens, and additionally, when released free in the mucosal environment, it can be a nutrient source for these bacteria. In healthy adults, approximately 300 μg of sialic acid is present per mg of colonic mucin. Elevated levels of sialic acid are induced by antibiotic therapy and pathogen infection. Elevated levels of free sialic acid in the gut, during and post antibiotic treatments, promote the expansion of strains of C. difficile and Salmonella that lack a sialidase (but contain a sialic acid catabolic operon) and thus rely on free sialic acid released from mucins by members of the gut microbiota (Ng K M, Ferreyra J A, Higginbottom S K, Lynch J B, Kashyap P C, Gopinath S, Naidu N, Choudhury B, Weimer B C, Monack D M, Sonnenburg J L. “Microbiota-liberated host sugars facilitate post-antibiotic expansion of enteric pathogens”. Nature. 502, 96-9 (2013)).

Cystic fibrosis (CF) patients battle life-long pulmonary infections with the respiratory pathogen Pseudomonas aeruginosa (PA). An overabundance of mucus in CF airways provides a favorable niche for PA growth. When compared with that of non-CF individuals, mucus of CF airways is enriched in sialyl-Lewisx, a preferred binding receptor for PA. Notably, the levels of sialyl-Lewisx directly correlate with infection severity in CF patients. Increased sialylation in airway mucins thus contribute to the severity of the infection (Jeffries J L, Jia J, Choi W, Choe S, Miao J, Xu Y, Powell R, Lin J, Kuang Z, Gaskins H R, Lau G W. Pseudomonas aeruginosa pyocyanin modulates mucin glycosylation with sialyl-Lewisx to increase binding to airway epithelial cells. Mucosal Immunol. 2015 Nov. 11. doi: 10.1038/mi.2015.119)

Therefore, reducing the amount of sialic acid in the mucosal environment is a novel strategy to reduce microbial infection.

Sialic acids constitute a structurally diverse family of nine-carbon acidic monosaccharides commonly found at the termini of the glycan chains on glycoproteins and glycolipids (Varki A., Nature. 2007 Apr. 26; 446(7139):1023-9). N-Acetylneuraminic acid (Neu5Ac), N-glycolylneuraminic acid (Neu5Gc), and 2-keto-3-deoxy-d-g/ycero-d-ga/acto-nonulosonic acid (KDN) are the three basic forms of sialic acids which are distinguished from one another by different substituents at carbon-5. Additional modifications include acetylation, lactylation, methylation, sulphation, for example, resulting in more than 50 structurally distinct sialic acids (Angata et al., Chem Rev. 2002; 102:439-469). As the outermost carbohydrate residues, sialic acids are critical recognition elements in a number of biologically important processes including cell-cell interaction, viral infection, tumour metastasis, as well as bacterial infections, as described above (Schauer R., Glycoconj J. 2000; 17:485-499; Chen & Varki, ACS Chem Biol. 2010 Feb. 19; 5(2):163-76).

For example, sialic acid residues found in terminal locations of mucins via α2-3/6 glycosidic linkages are prominent targets for commensal and pathogenic bacteria. The release of sialic acid by microbial sialidases allows the bacteria present in the mucosal environment to access free sialic acid for catabolism, decrypt host ligands for adherence, participate in biofilm formation, modulate immune function by metabolic incorporation, and expose the underlying glycans for further degradation (Lewis, A. L., Lewis, W. G., 2012. Cell. Microbiol. 14, 1174-1182; Juge N. Trends Microbiol. 2012 January; 20(1):30-9; Etzold S, Juge N. Curr Opin Struct Biol. 2014 October; 28:23-31; Juge et al. Biochem Soc Trans. 2016 February; 44(1):166-75; Ouwerkerk et al. 2013. Best Pract. Res. Clin. Gastroenterol. 27, 25-38; Tailford et al. Front Genet. 2015a Mar. 19; 6:81). However, microbial sialidases are poorly understood.

Synthetic methods enabling facile access to a wide variety of sialic acid derivatives are pivotal in sialobiology and drug discovery to explore and/or inhibit sialic acid-protein interactions. Chemo-enzymatic synthesis approaches are considered as attractive and practical strategies for the synthesis of sialosides, being any glycoside of sialic acid, including those containing uncommon sialic acid forms. However, limited protocols are available to date.

The present application describes the role that 2,7-anhydro-Neu5Ac may play in important biological roles in symbiotic interactions with the host and in the adaptation of gut symbionts to the mucosal environment. Previously 2,7-anhydro-Neu5Ac had been detected in rat urine (Schroder et al. (1983) in Glycoconjugates, Proceedings of the 7th International Symposium on Glycoconjugates (Chester, M. A., Heinegard, D., Lundblad, A., & Svensson, S., eds.) pp. 162-163 and human wet cerumen (ear wax) (Suzuki et al. J Biochem. 1985 February; 97(2):509-15).

However, the biological significance of naturally occurring 2,7-anhydro-Neu5Ac in body fluid and secretions is still largely unknown. This is due to the lack of an effective synthetic method for its production.

### SUMMARY OF THE INVENTION

Most of the microbial sialidases that have been identified to date are hydrolytic sialidases, which release free sialic acid from sialylated substrates.

A new class of intramolecular trans sialidases (ITS) has recently been identified from leech and from Streptococcus pneumonia. The present invention describes an ITS identified in gut commensal bacteria.

The present invention thus relates to a new enzyme from a gut commensal bacteria, Ruminococcus gnavus (R. gnavus), called an intramolecular trans-sialidase (“IT-sialidase”; “ITS” or “RgNanH”, the full amino acid sequence for which is set out in SEQ ID NO: 1 including a 25 amino acid signal sequence), which has recently been identified and characterised (Crost E H, Tailford L E, Le Gall G, Fons M, Henrissat B, Juge N. Utilisation of mucin glycans by the human gut symbiont Ruminococcus gnavus is strain-dependent. PLoS One. 8, e76341 (2013); Tailford L E., Owen C D, Walshaw J, Crost E H, Hardy-Goddard J, Le Gall G, de Vos W M, Taylor G L, Juge N. “Discovery of intramolecular trans-sialidases in human gut microbiota suggests novel mechanisms of mucosal adaptation”. Nature Comm 6, Article number: 7624 (2015)). R. gnavus is found in over 90% of healthy individuals and is one of the predominant species in the gut (Nature 2010 464:59-65; PLoS One. 9, e97279 (2014)). The enzyme has a 25 amino acid signal sequence (SEQ ID NO:4). The catalytic domain is also described herein (see FIG. 13). This enzyme cleaves off sialic acid from mucins, but in contrast to common sialidases expressed by gut bacteria, it releases a transglycosylation product, 2,7-anhydro-alpha-N-acetylneuraminic acid (2,7-anhydro-Neu5Ac), instead of free sialic acid. This unique feature provides IT-sialidase expressing commensal gut microbes such as R. gnavus with a competitive nutritional advantage, allowing bacteria to thrive within mucosal environments by scavenging sialic acid from host mucus (Tailford et al. (2015)) in a form, 2,7-anhydro-Neu5Ac, which may not be readily accessible to other bacteria including enteric and respiratory pathogens. Therefore, this enzyme can contribute to commensal/pathogen competition at the mucosal surface, by removing pathogen binding sites and producing a product that the pathogen cannot metabolise. This unusual activity has the potential to protect against any respiratory or gut pathogens that utilise sialic acid as a receptor or energy source. The IT-sialidase enzyme can reduce the availability of free sialic acid in the mucosal environment, therefore limiting outgrowth of these pathogens by effectively “starving” them. Additionally, the removal of (negatively charged) terminal sialic acids could alter the surface charge structure and viscosity of the mucus layer. Therefore the IT-sialidase could improve access for enzyme cocktails (mucinolytic therapy, in current clinical use in e.g. cystic fibrosis) whilst avoiding environmental enrichment for opportunist pathogens.

In one aspect of the invention there is provided an isolated amino acid sequence with 80% identity to the amino acid sequence as set out in SEQ ID NO:1 wherein said isolated amino acid sequence encodes an intramolecular trans-sialidase (IT-sialidase). Suitably sequence identity is measured over the whole length of the protein. In another aspect there is provided an isolated amino acid sequence with 80% identity to the amino acid sequence as set out in SEQ ID NO:3 wherein said isolated amino acid sequence encodes an intramolecular trans-sialidase (IT-sialidase). SEQ ID NO: 3 is the amino acid sequence for IT-sialidase, lacking the 25 amino acid signal sequence (SEQ ID NO:4) and comprises the catalytic domain (also referred to as RgNan NI or RgGH33 (SEQ ID NO:6)). In one embodiment, the amino acid sequence may have 80, 85, 90, 95 or 99% identity to the sequence set out in SEQ ID NO: 1, SEQ ID NO: 3 or SEQ ID NO: 6. References to IT-sialidase and its expression and use are to both the full length protein including the signal sequence and to a catalytic portion thereof.

While previously identified hydrolytic sialidases demonstrate a broad activity against α2,3-, α2,6- and α2,8-linked substrates, IT-sialidases such as the enzyme described herein tend to be specific for α2,3-linked substrates. Advantageously, the enzyme will only target/cleave off sialic acid bound via α2,3-glycosidic linkages. In mucins, sialic acid may be found attached via α2,3- or α2,6-glycosidic linkages, the linkage differences depend on the location e.g. respiratory tract vs gastro-intestinal tract, or even along the gastro-intestinal tract.

Thus, in one embodiment, an IT-sialidase in accordance with the invention has an enzymatic activity of producing 2,7-anhydro-Neu5Ac (2,7-anhydro-alpha-N-acetylneuraminic acid) from alpha2-3-linked sialic acid substrates. Advantageously, the product, 2,7-anhydro-Neu5Ac, is only utilised by some bacteria. Suitable methods for measuring enzyme activity are described herein. Suitably, said enzyme produces 2,7-anhydro-Neu5Ac selectively from alpha2-3-linked sialic acid substrates; for example, the enzyme recognises alpha2-3-linked sialic acid substrates and does not recognise alpha-2,6 linkages. Methods for determining the products of enzyme activity and substrate specificity are described herein. In one embodiment, peaks characteristic of 2,7-anhydro-Neu5Ac are detected using ¹H NMR. Examples of suitable alpha2-3-linked sialic acid substrates for use in an enzyme assay are shown in Table 3. In one embodiment, an alpha2-3-linked sialic acid substrate is mucin.

The characterisation and crystal structure of the active/catalytic site of an IT-sialidase in accordance with the invention is described herein. Accordingly, in another embodiment, the invention provides an IT-sialidase comprising any or all of the following amino acid motifs: an arginine triad made up of Arg257, Arg637, and Arg575; a catalytic pair formed by Glu559 and Tyr677; a general acid base, Asp282; and a pocket to accommodate the N-acetyl group between Ser364 and Ile338 (with reference to the amino acids as set out in SEQ ID NO:1). Suitably, the IT-sialidase has those amino acid residues 243-723 as set out in SEQ ID NO:1 which form the catalytic site. In another aspect there is provided a catalytic domain from an IT-sialidase. The generation and use of an isolated catalytic domain having the amino acids 243-723, designated RgNan-NI or GH33 is described herein.

In one embodiment, an IT-sialidase in accordance with the invention may be derived from bacteria of the gut bacteria. In one embodiment, the bacteria is derived from a bacteria of the Firmicutes phylum. Suitably, the IT-sialidase is derived from a Ruminococcus such as Ruminococcus gnavus (R. gnavus). In one embodiment, the IT-sialidase is R. gnavus ATCC 29149. In another embodiment, the IT-sialidase in accordance with the invention has the amino acid sequence set out in SEQ ID NO: 3.

In further aspects of the invention, there is provided an isolated nucleic acid molecule encoding an IT-sialidase in accordance with the invention, a vector comprising the nucleic acid and a host cell comprising said vector.

In one embodiment, the vector and host cell is suitable for protein expression and production. Suitable host cells, protein expression vectors and systems for protein production will be familiar to those skilled in the art and include those vectors and expression systems based on bacterial, fungal, yeast, baculovirus/insect and mammalian cells. In another embodiment, a vector may be suitable for gene delivery e.g. a viral (such as baculovirus, retrovirus, adenovirus), plasmids, artificial chromosomes or bacteriophage (such as lambda) vector.

In one embodiment, the host cell is a microorganism. Suitable microorganisms include bacteria, viruses or bacteriophage.

In another aspect, the invention provides an expression system for expressing an IT-sialidase comprising culturing a host cell in accordance with any aspect or embodiment of the invention. In another aspect, the expression system may comprise a vector in accordance with the invention.

Further provided is a method for the production of an IT-sialidase in accordance with the invention wherein said method comprises culturing the host cell under conditions suitable for expression of said nucleic acid molecule or vector to produce an IT-sialidase. In another aspect, said method comprises culturing an “IT-sialidase expressing microorganism” under conditions suitable for expression of said nucleic acid molecule or vector to produce an IT-sialidase. In one embodiment, the method comprises culturing Ruminococcus gnavus ATCC 29149. Suitable conditions for enzyme expression are described herein.

The term “intramolecular trans-sialidase expressing microorganism”, or “IT-expressing microorganism” or “IT-sialidase-expressing bacteria” as used herein refers to a both naturally occurring and non-naturally occurring microorganisms with express an IT-sialidase in accordance with the invention. Examples of naturally occurring IT-sialidase expressing microorganisms, predicted based on bioinformatics analyses, include naturally occurring and isolated bacterial strains such as those belonging to or derived from Ruminococcus or Lactobacillus strains. In one embodiment, an “IT-sialidase expressing microorganism” is Ruminococcus gnavus, preferably Ruminococcus gnavus ATCC 29149. In another embodiment, an IT-sialidase expressing microorganism is a host cell which has been transformed or transfected to comprise a nucleic acid molecule encoding the IT-sialidase in accordance with the invention. In a further embodiment, an IT-sialidase expressing microorganism may comprises a vector or expression system in accordance with the invention.

As described herein, an IT-sialidase in accordance with the invention can degrade mucin through enzymatic action on the sialic acid mucin carbohydrate chains. Enzymes that can degrade mucins may therefore form a new class of mucolytics. These enzymes may either break down the oligosaccharide side chains or the protein backbone of mucins.

Accordingly, the invention provides a treatment effective to alter mucin glycosylation by providing an IT-sialidase enzyme or an IT-sialidase-expressing bacterium. The specific glycosylation product, 2,7-anhydro-Neu5Ac, is only utilised by some bacteria thus giving those bacteria a competitive advantage over others. The invention further provides a method of treatment or prevention of infection by pathogens that utilise sialic acid as a receptor or energy source using IT-sialidase or an IT-sialidase expressing bacteria. In one embodiment, the pathogen is a respiratory or gut pathogen, such as a pathogenic bacteria. Accordingly, in another aspect there is provided a pharmaceutical composition comprising an IT-sialidase in accordance with the invention and one or more pharmaceutically acceptable carriers or excipients. Suitable pharmaceutically acceptable carriers or excipients for the delivery of an enzyme as a therapeutic will be familiar to those skilled in the art. In another aspect there is provided an IT-sialidase in accordance with the invention for use in therapy.

In another aspect there is provided a pharmaceutical composition comprising an IT-sialidase-expressing microorganism and one or more pharmaceutically acceptable carriers or excipients for use in therapy. Such a composition may be used as a therapeutic or prophylactic treatment. Suitable pharmaceutical carriers for use in administering a composition comprising microorganism such as an active bacteria will be familiar to those skilled in the art, particularly in the field of delivery of probiotics. Examples are described for example in WO2006079790, WO03053474, WO2014152338 and WO2008115061. Further examples are described below.

In one embodiment of any aspect of the invention, the IT-sialidase-expressing microorganism is a bacteria, suitably a bacteria derived from any bacterial strain which has Nan operon and IT sialidase. Suitably such bacteria can be selected from bioinformatics analysis of bacterial genomes. In one embodiment, a bacteria is selected from a Ruminococcus strain, such as Ruminococcus gnavus or a Lactobacillus strain, such as Lactobacillus salivarius. Suitably, the IT-sialidase expressing bacteria is Ruminococcus gnavus ATCC 29149. In another embodiment, the IT-sialidase expressing bacteria is Ruminococcus gnavus ATCC 35913.

In another embodiment, an IT-sialidase-expressing bacteria may be one which has been engineered to express IT-sialidase. Similarly, an IT-sialidase may be administered by providing an expression system in accordance with the invention. Administration of genetically modified bacteria as a treatment is described, for example, by Chen et al. Journal of Clinical Investigation, Vol. 124, 8, 3391-3406.

An IT-sialidase or an IT-sialidase-expressing microorganism in accordance with the invention may be for use in the treatment of respiratory disease or a gut infection. In this aspect, administering the trans-sialidase enzyme (IT-sialidase) either per se or in a host cell or expression system results in mucosal degradation and production of the product 2,7-anhydro-Neu5Ac that pathogenic bacteria cannot metabolise. Accordingly, this this would reduce the outgrowth of human pathogens, that is, reduce any competitive growth advantage of human pathogenic bacteria in comparison to non-pathogenic bacteria.

The invention therefore also provides a use of a pharmaceutical composition in accordance with the invention in the manufacture of a medicament for use in the treatment of a respiratory disease or a gut infection and a method for the treatment or prevention of a respiratory disease or a gut infection in a subject in need thereof comprising administering a therapeutically effective amount a pharmaceutical composition in accordance with the invention.

Respiratory diseases include Cystic Fibrosis, Idiopathic Pulmonary Fibrosis and Chronic obstructive pulmonary disease (COPD), asthma. For treatment of such respiratory diseases, a pharmaceutical composition or treatment would suitably be administered by inhalation e.g. in an aerosol or via a nebuliser. Suitable methods for administering and enzyme to the airways are described, for example in WO2015066664. For diseases such as Cystic Fibrosis, it may also be beneficial to administer a composition in accordance with the invention to the gut. The pharmaceutical composition may also be gaseous, comprising said IT-sialidase expressing bacteria for delivery with an aerosol, for example in the airways. Respiratory diseases may also involve infection with pathogenic bacteria such as Pseudomonas aeruginosa (PA). Accordingly, the invention also provides a composition or method of treatment of infection by such pathogenic bacteria.

The present invention provides a method to improve the access for enzyme cocktails to the mucus layer using an IT-sialidase or a microorganism expressing the enzyme. Accordingly, the present invention provides an adjunct therapy for existing treatments. For the treatment of Cystic Fibrosis, the enzyme may be administered in combination with an existing Cystic Fibrosis therapeutic. Suitable such therapeutics include those mucolytic (i.e. breaks down mucus) or mucinolytic (breaks down mucin, the particular protein constituent of mucus) therapeutics. Mucolytic compositions or therapeutics include Ambroxol, heparin, hypertonic saline, pulmozyme (rhDNase), mannitol, N-acetylcysteine and thiosachharide agents such as those described in EP2968215 and US2003/0087414. Suitable methods for using such therapeutics are described, for example in WO2011038901.

Accordingly in another aspect there is also provided a composition comprising an IT-sialidase or an IT-sialidase-expressing microorganism and a mucinolytic composition. In one embodiment, the composition comprises an IT-sialidase or an IT-sialidase expressing microorganism and a mucinolytic composition for separate, simultaneous or sequential administration. Further provided is a method of treatment of a respiratory disease in a subject in need thereof comprising administering a therapeutically effective amount of a) an IT-sialidase in accordance with the invention or IT-sialidase-expressing microorganism in accordance with the invention and b) a mucinolytic composition.

Gut infections include infections caused by pathogenic bacteria. Suitable gut pathogens for targeting with a pharmaceutical or food composition in accordance with the invention are any which utilise sialic acid as a receptor or energy source. In one embodiment, a gut infection may be iatrogenic enteritis or an infection by Salmonella, Clostridium e.g. Clostridium difficile. The present invention provides a preventative support therapy for iatrogenic enteritis in clinical care environments. Suitably, a method for the prevention of iatrogenic enteritis may include administration of a composition in accordance with the invention as a pill or formulation after an iatrogenic event or in combination with vaccines.

In another aspect of the invention, there is provided a food composition comprising an IT-sialidase or an IT-sialidase expressing bacteria and a suitable carrier. In one embodiment, the food composition is a probiotic formulation comprising an IT-sialidase expressing bacteria and a suitable carrier.

The pharmaceutical and food compositions may be liquid, comprising said IT-sialidase expressing bacteria, or solid, comprising dried said IT-sialidase or IT-sialidase expressing bacteria that can be reactivated when put in a suitable environment.

Said IT-sialidase or IT-sialidase expressing bacteria according to the invention may be dried by any system including freeze-dried or spray-dried and can contain suitable known adjuvants such as cryoprotectants. The preparation of the invention may be in the form of a tablet, a powder or similar form, containing a dose of said IT-sialidase or IT-sialidase expressing bacteria. The powder may then be mixed with solid food or foods with a high water-content, such as fermented milk products, for example yogurt, or reconstituted using a suitable liquid such as water, milk or similar drinkable liquids.

Alternatively, the dried preparation may be encapsulated in a suitable recipient to protect the IT-sialidase or IT-sialidase expressing bacteria during storage or during exposure to stomach acid. For administration of a pharmaceutical or food composition to the gut, the composition may be encapsulated, for example in microparticles or by an enteric coating. In particular, said IT-sialidase or IT-sialidase expressing bacteria composition may be encapsulated so that it is predominantly released in the gut. The dried preparation may also be encapsulated in a suitable recipient to control the release of said IT-sialidase or IT-sialidase expressing bacteria. In particular, encapsulation is desired when the invention is to be used in liquid or moist products to prevent the bacteria from growing and/or fermenting, and therefore from reducing its shelf-life. Suitable compounds for encapsulation for improving the shelf-life, as well as methods for carrying out the encapsulation are known in the art. Examples of encapsulation methods known in the art are presented in WO2012/101167 and in a review by Kaila Kailasapathy (Kailasapathy K. Microencapsulation of Probiotic Bacteria: Technology and Potential Applications. Curr. Issues Intest. Microbiol 3, 39-48 (2002)).

The preparations of the invention may further comprise all desired components, and/or additives which are suited for use in food or pharmaceuticals including flavours, colourings, preservatives, sugar, etc., as long as they do not affect the viability of said IT-sialidase expressing bacteria present therein.

In another embodiment, said food composition may be a probiotic formulation comprising said IT-sialidase expressing bacteria and a suitable carrier. The probiotic micro-organisms do not form part of any delivery system of IT-sialidase expressing bacteria. Examples of probiotic organisms that may be used are listed in WO2009/127519 and include yeasts such as Saccharomyces, Debaromyces, Kluyveromyces and Pichia, moulds such as Aspergillus, Rhizopus, Mucor and Penicillium and bacteria such as the genera Bifidobacterium, Propionibacterium, Streptococcus, Enterococcus, Lactococcus, Bacillus, Pediococcus, Micrococcus, Leuconostoc, Weissella, Oenococcus and Lactobacillus. Kluyveromyces lactis may also be used.

Suitable carriers may be an immediate-release carrier or a slow-release carrier and may comprise micro-crystalline cellulose (MCC), dextran, corn starch, flour, talc, sucrose, mannitol, lactose, calcium carbonate, polyvinylpyrrolidone (PVP), polyethylene oxide, hydroxypropyl methylcellulose (HPMC), hydroxypropylcellulose (HPC), polyvinyl alcohol (PVA) or the like.

The preparation can further contain prebiotic compounds, in particular specific fibres that produce butyrate/butyric acid or propionate/propionic acid upon fermentation; nitrogen donors such as proteins; and specific vitamins, minerals and/or trace elements. In one embodiment, a prebiotics approach will target the growth of IT-sialidase expressing bacteria already present in the gut.

As described herein, 2,7-anhydro-Neu5Ac binds to the active site of the sialidase. The ability of 2,7-anhydro-Neu5Ac to inhibit bacterial or viral enzymes such as neuraminidases (sialidases) suggests that 2,7-anhydro-Neu5Ac can be used as a therapeutic. Therefore, in another aspect there is provided a pharmaceutical composition comprising 2,7-anhydro-Neu5Ac (also known as “2,7-anhydro-N-acetylneuraminic acid” or “2,7-anhydroSA”; chemical structure shown in FIG. 3c and FIG. 8) and one or more pharmaceutically acceptable carriers or excipients. Another aspect provides 2,7-anhydro-Neu5Ac for use in therapy. Suitably, 2,7-anhydro-Neu5Ac or a pharmaceutical composition comprising 2,7-anhydro-Neu5Ac can be used for the treatment of a viral of bacterial infection. There is also provided a use of a pharmaceutical composition in accordance with any aspect of the invention in the manufacture of a medicament for use in the treatment of a viral or bacterial infection and a method for the treatment or prevention of a viral or bacterial infection in a subject in need thereof comprising administering a therapeutically effective amount of a pharmaceutical composition in accordance with any aspect of the invention. Suitable viral infections include infection by an influenza virus, for example. Suitable bacterial infections include infections by a pathogenic bacteria, and in particular a pathogenic bacteria which utilises sialic acid as a site of attachment or nutrient.

In another embodiment, 2,7-anhydro-Neu5Ac may be used in a prebiotic formulation. Suitable carriers for formulations are described above. For example, a composition comprising 2,7-anhydro-Neu5Ac may be formulated as a prebiotic for use in a formula milk supplement.

Sialic acid in the human milk is not in a free form but complexed within complex oligosaccharide structures, human milk oligosaccharides (HMOs). Sialylated HMOs are important to preserve gut health, with a protective role in e.g. immunity, inflammation and infection. The use of compounds such as 2,7-anhydro-Neu5Ac may promote growth of beneficial bacteria and inhibit adhesion of pathogens.

In another aspect, there is provided a method for production of a 2,7-anhydro sialic acid derivative comprising incubating an α2,3 linked sialic acid-containing substrate with an IT-sialidase, or an enzymatic fragment thereof, under conditions for enzymatic activity. Methods for detecting enzymatic activity are described herein.

Suitably the IT-sialidase, or an enzymatic fragment thereof, is in accordance with any aspect or embodiment of the invention. Thus, in one embodiment, the IT-sialidase is an IT-sialidase having an amino acid sequence with 80% identity to the amino acid sequence set out in SEQ ID NO:1, and having IT-sialidase activity. Suitably the IT-sialidase is derived from R. gnavus. In one embodiment, the IT-sialidase is derived from a specific strain of R. gnavus such as ATCC 29149 or ATCC 35913. In another embodiment, the IT-sialidase is RgNanH, as described herein. In another embodiment the IT-sialidase may be an enzymatic fragment thereof, by which is meant an enzymatically active fragment thereof. Suitably such a fragment may be a catalytic domain such as RgGH33, as described herein. Suitably such a fragment may have the amino acid sequence set out in SEQ ID NO:6 or be a functional homologue or fragment thereof.

Suitable sialic acid derivatives for production in the method of the present invention will be familiar to those skilled in the art along with suitable substrates for production of these derivatives, for examples of diverse sialic acid substrates see e.g. Angata et al., Chem Rev. 2002; 102:439-469. Suitably the 2,7-anhydro sialic acid derivative is 2,7-anhydro-Neu5Ac, 2,7-anhydro-Neu5Gc or 2,7-anhydro-KDN.

Suitable substrates include α2,3 linked sialic acid-containing substrates such as α2,3 linked Neu5Ac-containing substrates, including 4-Methylumbelliferyl-N-acetylneuraminic acid (4MU-Neu5Ac) (described, for example in U.S. Pat. No. 5,312,747) or Neu5Acα2-3Lac, for example, for the production of 2,7-anhydro-Neu5Ac; α2,3 linked Neu5Gc-containing substrates such as Neu5Gcα2-3Lac for the production of 2,7-anhydro-Neu5Gc; and α2,3 linked KDN-containing substrates such as KDN or Kdn-alpha2-3Lac for the production of 2,7-anhydro-KDN. Suitably, production of products may be monitored by NMR and electrospray ionisation mass spectrometry (ESI-MS).

Suitably the method in accordance with the invention may be carried out in a membrane enclosed environment, such as a membrane enclosed multiple enzyme (MEME) approach. Suitable methods are described herein. Advantageously, in this embodiment, the substrates are of sufficient size to remain within the membrane bag, such as glycoproteins. This facilitates purification of the product. Examples of suitable glycoprotein substrates include fetuin, Neu5Gc- or KDN-rich glycoproteins, or mucins.

In another aspect there is provided a method for production of 2,7-anhydro-Neu5Ac. A suitable method is described herein. Thus, a method comprising incubating an α2,3 linked sialic acid-containing substrate with an IT-sialidase in accordance with the invention or a catalytic domain derived therefrom, such as GH33, as described herein, under conditions for enzymatic activity is provided. Suitable α2,3 linked sialic acid-containing substrates for the production of 2,7-anhydro-Neu5Ac include fetuin as described in the Examples herein or its derivatives.

In one embodiment of any aspect of the invention, mucin may be used as a substrate. Mucin may be derived from any human or animal source. For example, bovine or porcine submaxillary mucin may be used as a substrate. In some embodiments, Neu5Gc-enriched glycoproteins such as Neu5Gc-rich submaxillary mucin, or KDN-enriched glycoproteins, may be used as substrates. In some embodiments of the invention these glycoproteins may be synthetically prepared, in other embodiments they may be harvested from natural sources.

In one embodiment, an IT-sialidase, or enzymatic fragment thereof, in accordance with the invention may be produced in a host cell such as Escherichia coli, for example, and purified before using with an α2,3 linked sialic acid-containing substrate.

In one embodiment, the method in accordance with any aspect of the invention may further comprise a subsequent incubation step. In one embodiment, the incubation of an α2,3 linked sialic acid-containing substrate with an IT-sialidase, or an enzymatic fragment thereof, under conditions for enzymatic activity is followed by incubation with a further enzyme, for example a sialic acid modifying enzyme such as a lyase or a sialic acid aldolase. The advantage of the second incubation step is to convert free sialic acid into smaller and uncharged enzymatic products which are more easily eliminated following the enzymatic reactions, producing a higher yield and greater purity of 2,7-anhydro-sialic acid product. Suitable sources of these sialic acid modifying enzymes will be familiar to those skilled in the art and include synthetic sources and bacterial expression systems.

In particular, the invention therefore provides a facile membrane enclosed multiple enzyme (MEME) approach for the preparative synthesis of 2,7-anhydro—Neu5Ac, 2,7-anhydro-Neu5Gc or 2,7-anhydro-KDN from glycoproteins using R. gnavus IT-sialidase (RgNanH), or an enzymatic fragment thereof, and a sialic acid aldolase-catalyzed reaction in one pot. Advantageously, the synthesis of 2,7-anhydro-Neu5Ac was achieved at high purity and in mg scale in laboratory conditions. Scale up from this is envisaged. Obtaining these products at a preparative scale is important for studying the biological importance of anhydro-2,7-sialic acid derivatives and their potential applications in the biomedical sector.

### FIGURES

FIG. 1. Kinetic analysis of the hydrolysis of sialylated substrates by RgNanH in presence or absence of inhibitors

Substrate (a) 4MU-Neu5Ac or (b) PNP-Neu5Ac was incubated with RgNanH (0.22 mM and 0.43 nM respectively) at 37° C., pH 6.5 and the release of product measured using a plate-reader. (c) Inhibition of RgNanH (1.00 nM) incubated with 4MU-Neu5Ac (0.51 mM) by zanamivir (●), Neu5Ac2en (▪), Siastatin B (▴) and oseltamivir carboxylate (♦), the rates are normalised to a % of the uninhibited rate. [I] is the inhibitor concentration. (d) 3′SL was incubated with RgNanH (2.25 nM) and the release of product measured by HPAEC-PAD. Experiments were done at least in duplicate (usually triplicate) and the error bars show the standard error of the mean.

FIG. 2. Specificity of RgNanH towards sialylated oligosaccharides

The enzymes were incubated with sialylated substrates at 37° C., pH 6.5 overnight and the reaction products were analysed by HPAEC-PAD. Control reactions without enzyme were also performed. The injection peak is marked (inj). Glycerol (gly) was also present in AkmNan0625 (a) Standards (Lac, Neu5Ac, 3′SL and 6′SL), (b) 3′SL, (c) 6′SL, (d) 3′-α-Sialyl-N-acetyllactosamine (3′SLacNAc). AkmNan1835 has a similar profile to AkmNan0635.

FIG. 3. H1 NMR analysis of RgNanH reaction products in presence of 4MU-Neu5Ac or 3′SL

RgNanH was incubated with (a) 4MU-Neu5Ac or (b) 3′SL at 37° C., pH 6.5 overnight and the reaction mixture analysed by ¹H NMR. Control reactions without enzyme (“−”, lower trace) were carried out in parallel with the reaction containing enzyme (“+”, upper trace). “A” corresponds to 2,7-anhydro-Neu5Ac, see (c) for chemical structure and NMR shifts.

FIG. 4. RgNanH catalytic domain bound to Neu5Ac2en

(a) Cartoon representation of the RgNanH NI-domain, the catalytic N-domain with secondary structure features is shown at the right/bottom right of each representation, and the inserted !-domain is shown with secondary structure features at the left/top left of each representation. Neu5Ac2en is shown as the globular structure bound in the active site. (b) The RgNanH NI-domain with an electrostatic surface applied, shown as shading. Neu5Ac2en is shown bound in the active site.

FIG. 5. The RgNanH active site bound in complex with 2,7-anhydro-Neu5Ac and sialidase inhibitors

Cartoon representation of the RgNanH active site complexed with (a) 2,7-anhydro-Neu5Ac, (b) Neu5Ac2en, (c) OC, and (d) Siastatin B. Bound ligands are shown in the centre with the unbiased Fo-Fc map contoured to σ=3. Relevant residues are highlighted as sticks, and water molecules as spheres. Black dashed lines represent the hydrogen-bonding network. The lower panel is related to the upper panel by a rotation along the z-axis of −90° followed by a rotation along the x-axis of −45°.

FIG. 6. Schematic representation of the nan locus in R. gnavus ATCC 29149

RUMGNA_02701 encodes a putative GDSL-like protein. RUMGNA_02700 encodes a putative sugar isomerase involved in sialic acid catabolism. RUMGNA_02699 encodes a protein with homology with transcriptional regulators of the AraC family. The following 3 genes code for a predicted solute-binding protein (RUMGNA_02698) and two putative permeases (RUMGNA_02697, RUMGNA_02696), components of a sugar ABC transporter. The following gene has homology with oxidoreductase from the Gfo/Idh/MocA family. The sialidase gene nanH (RUMGNA_02694) predicted to encode the GH33 enzyme comes next. Then nanE (RUMGNA_02693), which encodes a predicted ManNAc-6-P epimerase is followed by nanA (RUMGNA_02692) encoding a putative Neu5Ac lyase. nanK (RUMGNA_02691) is the last gene of the cluster, coding for a predicted ManNAc kinase.

FIG. 7. pH and temperature-dependence of RgNanH using 4MU-Neu5Ac as substrate

4MU-Neu5Ac (0.51 mM) was incubated in presence of 0.25 nM enzyme (filled circles) or in absence of enzyme (empty circles) as a control. The reaction was carried out in sodium phosphate buffer at different pHs (a) at 37° C., or at different temperatures (b) in phosphate buffered saline (PBS) at pH 7.4. The release of MU was measured by fluorimetry. The reaction was carried out in triplicate and the standard errors of the mean are shown.

FIG. 8. Structures of inhibitors used in this study

The chemical structures of the inhibitors used in this study (Neu5Ac2en, oseltamivir carboxylate, Siastatin B, zanamivir) are shown here with Neu5Ac and 2,7-anhydro-Neu5Ac included for comparison. Atoms are numbered in relation to in text references.

FIG. 9. Effect of Neu5Ac and Lac on RgNanH

4MU-Neu5Ac (0.51 mM) was incubated in presence of 1 nM enzyme, with the indicated concentration of Neu5Ac (0-0.3 mM) or Lac (0-1.5 mM). The reaction was carried out in sodium phosphate buffer at pH 6.5 and at 37° C. The release of MU was measured by fluorimetry.

FIG. 10. Reaction products of RgNanH and AkmNan0625 enzymes incubated with 4MU-Neu5Ac

The sialidases were incubated with 4MU-Neu5Ac at 37° C., pH 6.5 overnight and the reaction products analysed by ¹H NMR. “A” corresponds to 2,7-anhydro-Neu5Ac. Spectra similar to AkmNan0625 were obtained with AkmNan1835 (not shown).

FIG. 11. NMR Activity of RgNanH against sialylated glycoproteins

RgNanH was incubated with human α-glycoprotein (AGP) and fetuin (Fet) (or asialo-fetuin as a control) at 37° C., pH 6.5 overnight and the reaction mixture analysed by ¹H NMR.

FIG. 12. A wall-eyed stereo image of a portion of the 2Fo-Fc electron density map of the RgNanH 27-anhydro-Neu5Ac complex X-ray crystal structure

The map is contoured to 2.00 sigma.

FIG. 13. Shows a schematic of R. gnavus IT-sialidase (RgNanH), with the non-catalytic domain, CBM40 and the catalytic domain, GH33, showing the inserted domain.

FIG. 14. ESI(−)-MS spectra of 2,7-anhydro-Neu5Ac obtained using a) Fetuin+RgNanH (50 nM)) containing 17% of Neu5Ac; and b) Fetuin+RgNanH (50 nM)+sialic acid aldolase (20 U) containing 2% of Neu5Ac.

FIG. 15. 600 MHz NMR spectra of 2,7-anhydro-Neu5Ac obtained using a) Fetuin+RgNanH (50 nM) containing 17% of Neu5Ac; and b) Fetuin+RgNanH (50 nM)+sialic acid aldolase (20 U) containing 2% of Neu5Ac.

FIG. 16. The MEME protocol for the synthesis of 2,7-anhydro-sialic acid derivatives.

### DETAILED DESCRIPTION OF THE INVENTION

The human gut is populated with microorganisms which play important roles in health and disease (de Vos, W. M. & de Vos, E. A. J. Role of the intestinal microbiome in health and disease: from correlation to causation. Nutr Rev 70, S45-S56, (2012)). The majority of the gut bacteria are believed to reside within the intestinal mucus layer covering the gastrointestinal tract epithelial cells (Johansson, M. E., Larsson, J. M. & Hansson, G. C. The two mucus layers of colon are organized by the MUC2 mucin, whereas the outer layer is a legislator of host-microbial interactions. Proc Natl Acad Sci USA 108 Suppl 1, 4659-4665 (2011)).

Changes in the balance of different bacteria in our microbiota have been linked to inflammatory bowel diseases (IBD) (Berry, D. & Reinisch, W. Intestinal microbiota: A source of novel biomarkers in inflammatory bowel diseases? Best Pract Res Cl Ga 27, 47-58 (2013); Manichanh, C., Borruel, N., Casellas, F. & Guarner, F. The gut microbiota in IBD. Nat Rev Gastro Hepat 9, 599-608 (2012)). There is an emerging paradigm that mucus is critical in order to maintain a homeostatic relationship with our gut microbiota and that any deviation from this dynamic interaction has major implications for health (colitis, colorectal cancer, infections etc) (Johansson, M. E. V. et al. Composition and functional role of the mucus layers in the intestine. Cell Mol Life Sci 68, 3635-3641 (2011); McGuckin, M. A., Linden, S. K., Sutton, P. & Florin, T. H. Mucin dynamics and enteric pathogens. Nature reviews. Microbiol 9, 265-278 (2011); Pelaseyed, T. et al. The mucus and mucins of the goblet cells and enterocytes provide the first defense line of the gastrointestinal tract and interact with the immune system. Immunol Rev 260, 8-20 (2014); Sheng, Y. H., Hasnain, S. Z., Florin, T. H. & McGuckin, M. A. Mucins in inflammatory bowel diseases and colorectal cancer. J Gastroenterol Hepatol 27, 28-38 (2012)). For example, patients suffering from IBD have a disproportionate representation of mucin-degraders, such as Ruminococcus gnavus (Png, C. W. et al. Mucolytic bacteria with increased prevalence in IBD mucosa augment in vitro utilisation of mucin by other bacteria. Am J Gastroenterol 105, 2420-2428 (2010)), a common species of gut bacteria found in over 90% of people (Qin, J. J. et al. A human gut microbial gene catalogue established by metagenomic sequencing. Nature 464, 59-U70 (2010)).

In addition, the prominent terminal locations of host sialic acids have made them one of the most frequently targeted carbohydrate receptors for pathogen adherence. Respiratory pathogens, including Hemophilus influenzae, Streptococcus pneumoniae, and Pseudomonas aeruginosa share a common ecological niche, colonising the heavily sialylated secretions and surfaces of the upper respiratory tract. In cystic fibrosis, asthma, and a number of other severe respiratory conditions like chronic obstructive pulmonary disease, mucus hypersecretion and stasis exacerbate the ability of pathogens to colonise and multiply and increase risk of death (Venkatakrishnan V, et al. Expert Rev Respir Med 7, 553-576 (2013)). Polymorphism of the MUCSB mucin gene is a proven host susceptibility factor for idiopathic pulmonary fibrosis (IPF) and bacteria have recently been implicated in the pathogenesis and progression of IPF (Molyneaux P L et al. Am J Respir Crit Care Med 190, 906-913 (2014)).

Mucins are the most abundant protein in mucus, with a ‘bottle brush’ appearance of sialic-acid-capped O-glycan chains attached to the protein backbone via serine/threonine residues. Gel-forming mucins are secreted by goblet cells within the gastrointestinal tract, and represent the interface between the microbial community and host tissue (Ouwerkerk, J. P., de Vos, W. M. & Belzer, C. Glycobiome: bacteria and mucus at the epithelial interface. Best Pract Res Clin Gastroenterol 27, 25-38 (2013)). The prominent terminal locations of host sialic acids have made them one of the most frequently targeted carbohydrate receptors for pathogen adherence (Angata, T. & Varki, A. Chemical diversity in the sialic acids and related alpha-keto acids: An evolutionary perspective. Chem Rev 102, 439-469 (2002)). Microbes also express sialidases (also commonly referred to as neuraminidases), enzymes that cleave terminal sialic acid residues from host sialoglycan substrates. Bacterial sialidases and their mucosal sialoglycan targets contribute to host-microbe interactions at every mammalian mucosal surface. Sialidases have been proposed to promote bacterial survival in mucosal niche environments via (i) nutritional benefits of sialic acid catabolism, (ii) unmasking of cryptic host ligands used for adherence, (iii) participation in biofilm formation and (iv) modulation of immune function (Lewis, A. L. & Lewis, W. G. Host sialoglycans and bacterial sialidases: a mucosal perspective. Cell Microbiol 14, 1174-1182 (2012)). Moreover, removal of sialic acid from sialomucin exposes the glycan moiety that can be rapidly catabolised (Ouwerkerk et al. (2013)). A number of sialidase-expressing microbes benefit from sialic acid hydrolysis via catabolism and utilisation of sialic acid as a carbon source.

In bacteria, the genes involved in sialic acid metabolism are usually found clustered together forming what is denominated as a Nan cluster. The canonical cluster nanA/K/E, first described for Escherichia coli (Plumbridge, J. & Vimr, E. Convergent pathways for utilisation of the amino sugars N-acetylglucosamine, N-acetylmannosamine, and N-acetylneuraminic acid by Escherichia coli. J Bacteriol 181, 47-54 (1999)) involves genes encoding the enzymes N-acetylneuraminate lyase (NanA), kinase (NanK) and N-acteylmannosamine (ManAc) epimerase (NanE), converting sialic acid (Neu5Ac) into N-acetylglucosamine-6-P (GlcNAc-6-P) whereas the genes encoding NagA (GlcNAc-6-P deacetylase) and NagB (glucosamine-6-P deaminase) converting GlcNAc-6-P into fructose-6-P (Fru-6-P), which is a substrate in the glycolytic pathway, vary in their locations among the different genomes that encode the Nan cluster (Vimr, E. R., Kalivoda, K. A., Deszo, E. L. & Steenbergen, S. M. Diversity of microbial sialic acid metabolism. Microbiol Mol Biol Rev: MMBR 68, 132-153 (2004)). An alternative pathway for sialic acid metabolism has later been discovered in Bacteroides fragilis, relying on the action of an aldolase (NanL), a novel ManNAc-6-P epimerase (also named NanE; we refer to this as NanE2 to distinguish it from the E. coli NanE which we refer to as NanE1) and a hexokinase (RokA), converting Neu5Ac into GlcNAc-6-P, an intermediate in the common pathway for aminosugar utilisation (Brigham, C. et al. Sialic acid (N-acetyl neuraminic acid) utilization by Bacteroides fragilis requires a novel N-acetyl mannosamine epimerase. J Bacteriol 191, 3629-3638 (2009)). This novel sialic utilisation pathway is defined by the nanLET cluster, where NanT is a transport gene for sialic acid (Brigham et al. (2009); Severi, E., Hood, D. W. & Thomas, G. H. Sialic acid utilisation by bacterial pathogens. Microbiology 153, 2817-2822 (2007)). The majority of the bacteria that encode either of these Nan clusters colonise mucus regions of the human body, such as the gut, lung, bladder or oral cavity, where sialic acid is highly abundant and can serve as a source of energy, carbon, and nitrogen (Lewis et al. (2012); Almagro-Moreno, S. & Boyd, E. F. Insights into the evolution of sialic acid catabolism among bacteria. BMC Evol Biol 9, 118 (2009)). Interestingly, some bacteria appear to have only partial packages of enzymes for scavenging host sialic acids. For example, Bacteroides thetaiotaomicron encodes a sialidase and can release free sialic acid, but lacks the Nan operon required to consume the liberated monosaccharide and does not appear capable of consuming hydrolysed sialic acids (Marcobal, A. et al. Bacteroides in the infant gut consume milk oligosaccharides via mucus-utilization pathways. Cell Host Microbe 10, 507-514 (2011)). On the other hand, Salmonella typhimurium and Clostridium difficile encode the Nan operon but each lacks the sialidase (Almagro-Moreno et al. (2009)), and have been suggested to rely on other sialidase-producing organisms to acquire this potential nutrient source (Vimr et al. (2004); Ng, K. M. et al. Microbiota-liberated host sugars facilitate post-antibiotic expansion of enteric pathogens. Nature 502, 96-99 (2013)).

It was shown that R. gnavus ATCC 29149 encodes a complete Nan cluster (NanA/K/E) as well as the sialidase required for sialic acid liberation from host sialylated substrates (NanH) and a putative sialic acid ABC transporter (FIG. 6) and that the whole cluster was induced when the cells were grown in the presence of α2-3 linked sialic acid lactose (3′SL) or mucin (Crost, E. H. et al. (2013)). The sialidase and Nan cluster is absent from the genome R. gnavus E1, which is unable to grow on mucin as a sole carbon source (Crost et al. (2013)). Taken together, these data indicate that sialic acid metabolism is key to the ability of R. gnavus strains to utilise mucin as a nutrient source, which is in agreement with earlier studies on mucin degradation in the human colon ecosystem (Corfield, A. P, et al. Mucin degradation in the human colon: production of sialidase, sialate O-acetylesterase, N-acetylneuraminate lyase, arylesterase, and glycosulfatase activities by strains of fecal bacteria. Infect Immun 60, 3971-3978 (1992); Hoskins, I. C., et al. Mucin degradation in human colon ecosystems. Isolation and properties of faecal strains that degrade ABH blood group antigens and oligosaccharides from mucin glycoproteins. J Clin Invest 75, 944-953 (1985); Hoskins, I. C., et al. Mucin glycoprotein degradation by mucin oligosaccharide-degrading strains of human faecal bacteria. Characterisation of saccharide cleavage products and their potential role in nutritional support of larger faecal bacterial population. Microb Ecol Health Dis 5, 193-207 (1992)). However, the bacteria (ATCC 29149 and E1) could not grow on sialic acid as carbon source (Crost et al. (2013)).

The functional and structural characterisation of R. gnavus ATCC 29149 sialidase (RgNanH; having the amino acid sequence set out in SEQ ID NO:1) is reported herein. It is also demonstrated herein that the enzyme is an intramolecular trans-sialidase (IT-sialidase) producing 2,7-anhydro-Neu5Ac selectively from α2-3 linked sialic acid substrates, the first one reported in a gut commensal microbe suggesting an unprecedented mechanism underpinning adaptation of gut bacteria to the sialylated-rich mucosal environment. IT-sialidase expressing bacteria also have the ability to prevent antimicrobial resistance by reducing the levels of free sialic acid in the gut.

Enteric pathogens such as Salmonella typhimurium and Clostridium difficile are the leading cause of hospital-acquired diarrhoea, ranging from mild cases to severe pseudo-membranous colitis, collectively known as C. difficile infection (CDI). In the UK alone there are approximately 25,000 cases of CDI each year. 17% of patients diagnosed with CDI die within 30 days. The standard treatment for CDI is metronidazole or vancomycin for severe CDI. In humans, the use of antimicrobials is a major risk factor for CDI.

In addition to increasing resistance, oral antimicrobial therapy is one of the leading risk factors for diseases associated with Salmonella and C. difficile, disrupting the normal intestinal microbiota and creating conditions that favor acquisition and proliferation of these pathogens. The technology developed in the present invention can be deployed as a preventative support therapy for iatrogenic enteritis in clinical care environments. Recurrence CDI occurs in about 20% of patients. Current therapies range from stronger antibiotics (effective but prone to relapse on withdrawal) to faecal transplantation to restore the normal microbiota. The use of IT-sialidase expressing bacteria (commensals or probiotics) given to all patients undergoing antibiotic therapy to prevent CDI from occurring represents a more attractive strategy. Routine antimicrobial therapy is not recommended for mild or moderate cases of Salmonellosis so alternative treatment such as the one of the present invention will reduce the risk of antimicrobial resistence.

The present invention can also be used as adjunct therapy for interventions targeting respiratory infections. In cystic fibrosis, asthma, and other severe respiratory conditions like chronic obstructive pulmonary disease, mucus hypersecretion and stasis exacerbate the ability of pathogens to colonise and multiply and increases risk of death. Respiratory pathogens e.g. Hemophilus influenzae, Streptococcus pneumoniae, and Pseudomonas aeruginosa share a common ecological niche, attaching to sialylated structures of the upper respiratory tract. Using IT-sialidase (in aerosol) removes attachment sites for these opportunistic pathogens. Furthermore, the removal of terminal sialic acids alters the charge and viscosity of mucus. Therefore the treatment can improve access for mucinolytics whilst avoiding environmental enrichment by opportunist pathogens.

In summary, IT-sialidase expressing bacteria have the ability to reduce C. difficile and Salmonella mucosal expansion by limiting free sialic acid availability in the gut. This technology has the potential to protect against any respiratory or gut pathogens that utilise sialic acid as a receptor or energy source.

Accordingly, the invention relates to said IT-sialidase expressing bacteria for use in medicine, in particular for the therapeutic or prophylactic treatment of antimicrobial resistance, iatrogenic enteritis, irritable bowel syndrome, cystic fibrosis, asthma, and other severe respiratory conditions like chronic obstructive pulmonary disease. The invention further relates to the use of IT-sialidase expressing bacteria as preventative support therapy for iatrogenic enteritis in clinical care environments or as an adjunct therapy for mucinolytic interventions.

The present invention also relates to a method for the production of 2,7-anhydro-modified sialic acid derivatives. The choice of substrate to be incubated in the presence of an enzyme with IT-sialidase activity in accordance with the present invention determines the particular 2,7-anhydro-modified sialic acid derivative produced. The present invention also represents a convenient and efficient membrane enclosed multienzyme (MEME) approach for producing 2,7-anydro-modified sialic acids in a pure form, starting from readily available glycoproteins. The MEME protocol is exemplified in FIG. 16. As the occurrence of 2,7-anhydro-sialic acid in the gut has only recently been reported, as described herein, the impact it may have on gut microbiota and human health is, as yet, unexplored. The synthetic methods described herein offer general and straightforward access to a class of sialic acid derivatives and show promise to assess the biological significance and potential applications of 2,7-anydro-modified sialic acids in the context of drug discovery and biomedical research.

### EXAMPLES

### Example 1—Identification and Characterisation of RgNanH (intramolecular trans-sialidase)

### Methods

Reagents

All chemicals were obtained from Sigma (St. Louis, US) unless otherwise stated. The Akkermansia muciniphila sialidases, termed here AkmNan0625 and AkmNan1835, were obtained by His-Tag overexpression of their genes (Amuc_0625 and Amuc_1835, respectively (van Passel, M. W. et al. The genome of Akkermansia muciniphila, a dedicated intestinal mucin degrader, and its use in exploring intestinal metagenomes. Plos One 6, e16876 (2011)) in E. coli BL21(DE3) and subsequent purification to homogeneity by immobilised metal affinity chromatography (IMAC) as described below. The sialylated oligosaccharides and OC were obtained from Carbosynth (Berks, UK).

Cloning, Expression and Purification of Full-Length Sialidases

The full length RgNanH sialidase excluding the signal sequence (residues 1-25) was cloned into the pOPINF expression system (Berrow, N. S. et al. A versatile ligation-independent cloning method suitable for high-throughput expression screening applications. Nucleic Acids Res 35 (2007)), introducing an His-tag at the N-terminus. The primers used were; Forward AAGTTCTGTTTCAGGGCCCGCAAGAGGCCCAGACAGAT, Reverse ATGGTCTAGAAAGCTTTATGGTTGAACTTTCAGTTCATC. DNA manipulation was carried out in E. coli XL1 Blue (New England BioLabs, Boston, US). Sequences were verified by DNA sequencing by Eurofins (Ebersberg, Germany). E. coli BL21 (New England BioLabs) cells were transformed with the recombinant plasmid harbouring the sialidase gene according to manufacturer's instructions. For small scale expression the recombinant cells were grown to an OD₆₀₀ of 0.6 in Luria-Bertani Broth (LB) (10-50 mL) and then induced with 1 mM Isopropyl β-D-1-thiogalactopyranoside (IPTG) overnight at 22° C. The cells were harvested by centrifugation at 10 000 g for 20 min. Large scale expression (1 L) was carried out in “Terrific Broth Base with Trace Elements” autoinduction media (ForMedium™, Dundee, UK) growing cells for 3 h at 37° C. and then induced at 16° C. for 48 h. The cells were harvested by centrifugation at 10 000 g for 20 min. The His-tagged proteins were purified by IMAC. His-Bind™ resin was used according to the manufacturer's instructions (Novagen, Darmstead, Germany). Fractions containing the sialidase or domains were dialysed against 2×4 L 10 mM HEPES pH 8.0 and concentrated using a 10 kDa MWCO Vivaspin column (Vivaspin, Goettingen, Germany) up to 6 mg/mL. The protein was further purified by gel filtration using the Superdex 200 column on an Akta system (GE Health Care Life Sciences, Bucks., UK). Protein purification was assessed by standard SDS-PAGE using the both the NuPAGE Novex 4-12% Bis-Tris (Life Technologies, Paisley, UK) and RunBlue 12% SDS-PAGE gels (Expedeon, Cambs, UK). Protein concentration was measured with a Nanodrop (Thermo Scientific, Wilmington, USA) and using the extinction coefficient calculated by Protparam (ExPASy-Artimo, 2012) from the peptide sequence.

Activity Assays and Kinetics

The purified enzymes were incubated with the substrate in buffer made from 20 mM Na₂HPO₄ and 20 mM NaH₂PO₄ adjusted to a pH of pH 6.5 containing 1 mg/mL BSA at 37° C. unless otherwise stated. For 4MU-Neu5Ac, the progression of the reaction was monitored by the release of the MU using a 96-wells plate reader (BMG Labtech, Ortenberg, Germany) using fluorescence with an excitation at 340 nm and an emission at 420 nm. For determining the optimal temperature the assay was carried out in 20 mM phosphate buffered saline (PBS) at pH 7.4. For determining the optimal pH the assay was carried out at 37° C. using buffer made from 20 mM Na₂HPO₄ and 20 mM NaH₂PO₄ in varying proportions to obtain the desired pH. PNP release from PNP-Neu5Ac was monitored in a similar way measuring absorbance at 405 nm every 60 sec. For 3′SL, the reaction was monitored by High Performance Anion Exchange Chromatography-Pulsed Amperometric Detection (HPAEC-PAD). An aliquot of the reaction was removed from the main reaction volume at the reaction stopped by boiling for 20 min, the enzyme was then removed by centrifugation at 17 000 g for 10 min and filtration with a 0.2 μm filter (Millipore, Billerica, US). The sugars were separated by HPAEC with an isocratic gradient of 100 mM NaOH, 100 mM NaAC at 1 mL/min on a CarboPac PA1 protected with a guard column and detected using PAD on a Dionex ICS5000 system (Thermo Scientific, Hemel Hempstead, UK). The column was cleaned with 10 mL of 500 mM NaOH, 500 mM NaAc and the column reequilibrated with 100 mM NaOH, 100 mM NaAc. An internal standard of fucose was used in order to quantify the results.

For 4MU-Neu5Ac and 3′SL, the rate of hydrolysis without enzyme was not significant so raw data were used. However, for pNP-Neu5Ac the rate of hydrolysis without enzyme was significant so the rate with enzyme was corrected by the subtraction of the rate without enzyme at each concentration of substrate. For 3′ SL, the rate of reaction was calculated by plotting the amount of substrate remaining over the course of >50% of the reaction using the following equation (Matsui, I. et al. Subsite Structure of Saccharomycopsis Alpha-Amylase Secreted from Saccharomyces-Cerevisiae. J Biochem-Tokyo 109, 566-569 (1991));

kt=ln([S₀]/[Sₜ])

where k is k_{cat}/K_{M}×enzyme concentration, t is time, [S₀] and [Sₜ] are substrate concentrations at time 0 and t, respectively.

Kinetic data were obtained from at least duplicate (usually triplicate) experiments, and the kinetic parameters were calculated by fitting the initial raw data to the Michaelis-Menten equation using a non-linear regression analysis program (Prism 6, GraphPad, San Diego, USA), error bars (standard error of the mean) are shown.

For specificity tests against sialylated oligosaccharides (1 mM) and glycoproteins (1 mg/ml) 1 nM of enzyme was incubated with the substrate at 37° C., pH 6.5, overnight. For inhibition studies the enzyme and inhibitor were pre-incubated together for 15 min at 37° C. and the reaction started by the addition of the substrate, the IC₅₀ values were determined using Prism 6.

NMR Analysis

Extracts (400 μL) were mixed with 200 μL of D₂O and 20 μL of a solution of D₂O containing 1 mM of TSP (sodium 3-(trimethylsilyl)-propionate-d4). Samples (500 μL) were transferred into a 5-mm NMR tube for spectral acquisition. The ¹H NMR spectra were recorded at 600 MHz on a Bruker Avance spectrometer (Bruker BioSpin GmbH, Rheinstetten, Germany) running Topspin 2.0 software and fitted with a cryoprobe and a 60-slot autosampler. Each ¹H NMR spectrum was acquired with 128 scans, a spectral width of 8,012.8 Hz, an acquisition time of 2.04 s, and a relaxation delay of 2.0 s. The “noesypr1d” presaturation sequence was used to suppress the residual water signal with a low-power selective irradiation at the water frequency during the recycle delay and a mixing time of 100 ms. Spectra were transformed with a 0.3-Hz line broadening, manually phased, baseline corrected, and referenced by setting the TSP methyl signal to 0 ppm.

Protein Production and Crystallisation

The RgNanH NI-domain (residues 237-723) was cloned into the pEHISTEV vector (Liu, H. T. & Naismith, J. H. A simple and efficient expression and purification system using two newly constructed vectors. Protein Expres Purif 63, 102-111 (2009)) using the primers: Forwards, GATATCGGATCCAATATCTTTTATGCAGGAGATGC; Reverse, TGGTGCTCGAGTTTATGGTTGAACTTTCAGTTCATC. The domain was expressed and purified as described above but with the additional step of removal of the 6-histidine affinity tag by Tobacco Etch Virus (TEV) protease at a mass ratio of 1:50 overnight at 4° C. High purity fractions were pooled and concentrated to 36 mg/ml for storage and crystallisation trials.

All crystallisation experiments were carried out at 20° C. by the sitting drop, vapour diffusion method. Initial conditions were screened by the high-throughput Gryphon system (Art Robbins). Optimization was carried out manually with extensive use of serial micro-seeding. The best conditions for N-domain crystallisation were: 0.8 M NaH₂PO₄, 1.2M K₂HPO₄, sodium acetate 0.1M pH 4.5 (condition 1); 1.2 M NaH₂PO₄, 0.8 M K₂HPO₄, 0.1 M CAPS pH 10.5 (condition 2); and 12.5% PEG 3350 200 mM calcium chloride (condition 3). Typically the crystallisation drop consisted of 1 μl protein solution at 10-20 mg/ml, 1 μl reservoir solution and 0.25 μl seed stock solution prepared according to Bergfors and collaborators (Bergfors, T. Seeds to crystals. J Struct Biol 142, 66-76 (2003)). Crystals generally appeared after 48 hours and grew to full size in 72 h.

Ligand protein complexes were achieved by adding ligand stock solution in H₂O directly to the crystallisation drop. The crystallisation conditions and final ligand concentrations for the relevant complexes were: Neu5Ac2 en, 15 mM for 20 min in condition 1; Siastatin B, 6 mM for 20 min in condition 2; OC, 10 mM for 240 min in condition 3. To achieve the 2,7-anhydro-Neu5Ac complex the crystals were soaked with 20 mM 3′SL for 30 min in condition 1. Crystals were cryoprotected by stroking the crystal, using a nylon loop, across the top of a 1 μl drop containing the crystallisation solution with 25% glycerol.

Structure Determination and Refinement

Data were collected in-house at 100 K on a Rigaku 007HFM rotating anode X-ray generator with a Saturn 944 CCD detector at a wavelength of 1.54178 Å. The data were processed with HKL2000 (Otwinowski, Z. & Minor, W. in Methods in Enzymology Vol. 276 (ed Jr. & R. M. Sweet C. W. Carter, Eds.) Ch. Macromolecular Crystallography, 307-326 (Academic Press, 1997)). The phase problem was solved by molecular replacement using PHASER (Mccoy, A. J. et al. Phaser crystallographic software. J Appl Crystallogr 40, 658-674 (2007)) using the structure of NanL catalytic domain (PDB 2SLI) as a search model. This was followed by manual rebuilding with COOT (Emsley, P., Lohkamp, B., Scott, W. G. & Cowtan, K. Features and development of Coot. Acta Crystallogr D 66, 486-501 (2010)) and refinement using REFMACS (Murshudov, G. N. et al. REFMACS for the refinement of macromolecular crystal structures. Acta Crystallogr D 67, 355-367 (2011)). The RgNanH-Siastatin B made use of the Buccaneer pipeline (Cowtan, K. The Buccaneer software for automated model building. 1. Tracing protein chains. Acta Crystallogr D 62, 1002-1011 (2006)). Ramachandran statistics in the form outliers (%)/favoured (%): 2,7-anhydro-Neu5Ac complex, 0.00/97.1; Neu5Ac2en complex 0.00/96.3; OC complex, 0.00/96.5; Siastatin B, 0.41/95.9. For a stereo view of a portion of electron density, see FIG. 12. All molecular graphics were generated with PYMOL (Schrödinger, L. L. C. The {PyMOL} Molecular Graphics System, Version˜1.3r1 (2010)).

Genomic Analysis of Bacterial IT-Sialidase

We searched for Nan clusters, sialidase-encoding genes and other associated genes in 8,236 NCBI-distributed genomes (ftp://ftp.ncbi.nlm.nih.gov) that had annotations of protein-coding sequences and their products available (May 2014), as well as two additional R. gnavus genomes (Hoskins et al. (1985); Cervera-Tison, M. et al. Functional Analysis of Family GH36 alpha-Galactosidases from Ruminococcus gnavus E1: Insights into the Metabolism of a Plant Oligosaccharide by a Human Gut Symbiont. Appl Environ Microbiol 78, 7720-7732 (2012)). These represent 8,126 unique strains of around 2,800 species, comprising 28.6 million protein sequences. We used HMMER3 (http://hmmer.org) to query profile Hidden Markov Models (pHMMs) of the relevant protein domain sequences; more details are in the Bioinformatics Analysis section. Briefly, we refer to the 7 relevant protein domains as follows: (i) NanA (R. gnavus NanA and homologues including B. fragilis NanL), (ii) NanK (RgNanK and homologues), (iii) NanE1 (RgNanE and homologues including E. coli NanE), (iv) NanE2 (B. fragilis NanE and homologues), (v) ‘Sialidase’ (the sialidase domain of RgNanH and homologues but excluding the I-domain), (vi) I-domain (of RgNanH and homologues where present), (vii) CBM40 (of RgNanH and homologues where present). We used pHMMs from Pfam (Finn, R. D. et al. Pfam: the protein families database. Nucleic Acids Res 42, D222-D230 (2014)) when appropriate, but it was necessary to construct our own for (v, vi), respectively from 984 and 132 sequence segments from the GH33 family (www.cazy.org). We also built our own model (vii) from 36 segments of these sequences, although this performed very similarly to a Pfam pHMM; note that this Pfam domain (PF02973) is named ‘Sialidase’ but corresponds to CBM40, and not to the actual Sialidase domain (v). Note also that (v) detects conventional GH33 domains as a contiguous match as well as RgNanH-type domains as a segmented match with a gap representing the location of the I-domain matched by (vi). Having located the gene loci encoding the proteins matching our pHMMs, we defined gene clusters as cases of all genes of interest (NanA/K/E) being contained within 15 consecutive loci (which may include intervening genes). In all cases we ignored all hits with an independent E-value of >10⁻⁴.

Metagenomic Analysis of IT-Sialidase Prevalence

We analysed gut metagenomic data published by the MetaHIT consortium in a study of patients diagnosed with inflammatory bowel disease (IBD) and a control group (Qin et al. (2010)). We translated the assembled coding sequences (obtained from ftp://public.genomics.org.cn/BGI/gutmeta/SingleSample_GenePrediction) of the metagenomes of the 125 human subjects (99 with no IBD, 25 with IBD) described in Table 6 of reference prior to searching for pHMMs (v, vi) using HMMER3 as before. Further details are in the Bioinformatics Analysis section.

Bioinformatics Analysis

We searched all available genomes distributed by the NCBI (ftp://ftp.ncbi.nlm.nih.gov) for Nan clusters and “sialidase-encoding genes”, as follows. We downloaded data (May 2014) for 2,771 complete (URI: /genomes/Bacteria) and 6,977 draft (/genomes/Bacteria_DRAFT) bacterial genomes, as well as 4,549 plasmid sequences (/genomes/Plasmids). These data included one R. gnavus strain (ATCC 29149) but we supplemented this with corresponding data from four other strains: 0055_001C and AGR2154 (both available at http://www.ncbi.nlm.nih.gov/genome/genomes/979 but omitted from the FTP distribution), E1 (Otwinowski, Z. & Minor, W. in Methods in Enzymology Vol. 276 (ed Jr. & R. M. Sweet C. W. Carter, Eds.) Ch. Macromolecular Crystallography, 307-326 (Academic Press, 1997)) and ATCC 35913 (Mccoy, A. J. et al. Phaser crystallographic software. J Appl Crystallogr 40, 658-674 (2007)). We used the provided annotations of protein-coding sequences and their products. A total of 8,238 genomes had available protein sequence data; these represented 8,126 unique strains of around 2,800 species. We searched all 28.6 million protein sequences for the presence of the domains of interest, and related these to the genomic locations of their corresponding genes for the purpose of determining gene clusters. Note that in cases where the sequence data are highly fragmented (due to genes occurring on different DNA fragment sequences, or occurring in missing genomic regions), the analysis of draft genomes may include some false negatives. We used profile Hidden Markov Models (pHMMS) of each protein domain as search queries, using HMMER3 version 3.1b1 (http://hmmer.org), using a maximum independent E-value of 10⁻⁴. Where possible, we used available pHMMs from the Pfam database (Emsley, P., Lohkamp, B., Scott, W. G. & Cowtan, K. Features and development of Coot. Acta Crystallogr D 66, 486-501 (2010)); we verified their suitability by comparing performance on test data (not shown) with pHMMs constructed from our own alignments of homologues of R. gnavus sequences. This confirmed that Pfam domains (i) PF00701 (“DHDPS”), (ii) PF00480 (“ROK”), (iii) PF04131 (“NanE”), and (iv) PF07221 (“GlcNAc_2-epim”) were very suitable models respectively for (i) R. gnavus NanA (encoded by RUMGNA_02692) and homologues (including B. fragilis NanL); (ii) R. gnavus NanK (RUMGNA_02691) and homologues; (iii) R. gnavus NanE (RUMGNA_02691) and homologues (including E. coli NanE (Plumbridge et al. (1999))); (iv) B. fragilis NanE (Brigham et al. (2009)) and homologues (this NanE is an epimerase homologous to renin-binding proteins). For clarity, we refer to these four domains as NanA, NanK, NanE1, NanE2. For NanH and homologues, we built our own model from an alignment of RgNanH (RUMGNA_02694) and 984 sequences from the GH33 family (www.cazy.org); this alignment was edited to remove N- and C-terminal segments which are absent from RgNanH, as well as a segment corresponding to the I-domain. This model (which we refer to as “Sialidase”) therefore detected both conventional GH33 domains as a contiguous match, and also RgNanH-type domains as a segmented match with a gap representing the location of the I-domain. We built a model of the I-domain itself from the corresponding segments in the CAZy GH33 sequences (132 sequence segments). Note that the Pfam domain PF02973 named “Sialidase” appears to correspond to the carbohydrate-binding module CBM40 (www.cazy.org), so we did not use this to search for NanH. However, we built our own model named “CBM40”, from 36 sequence segments from the CAZy database, corresponding to the RgNanH CBM40 domain; this performed similarly to PF02973. Gene clusters: we defined clusters as N consecutive gene loci where N is the smallest number such that all genes of interest are present in any order, and N≤Nₘₐₓ, the largest permitted cluster size. We found that the number of genomes positive for a cluster of NanA, NanK, and NanE1 effectively plateaued at Nₘₐₓ=15, so we used that as the maximum size (87% of all genomes with these three genes on the same genomic sequence fulfil N≤15). Human metagenome data: we translated the assembled coding sequences of the metagenomes of the 125 subjects (99 with no IBD, 26 with IBD) described in Table S1 of reference (Qin et al. (2010)) (MetaH IT Consortium study) prior to searching with HMMER3 as before. We found this method to be much more sensitive than BLASTP (or TBLASTN on the coding sequences themselves) searches even with a liberal BLAST E-value cut-off of 10⁻³. Manual inspection of a sample of the additional hits arising from HMMER3 compared to BLAST indicated that they were clearly homologous to the query sequences. Analyses and post-processing were automated by Perl scripts, including some BioPerl modules (Stajich J. E. et al. Genome Res 12(10), 1611-8 (2002)).

### Results

RgNanH Produces 2,7-Anhydro-Neu5Ac from α2-3 Linked Neu5Ac

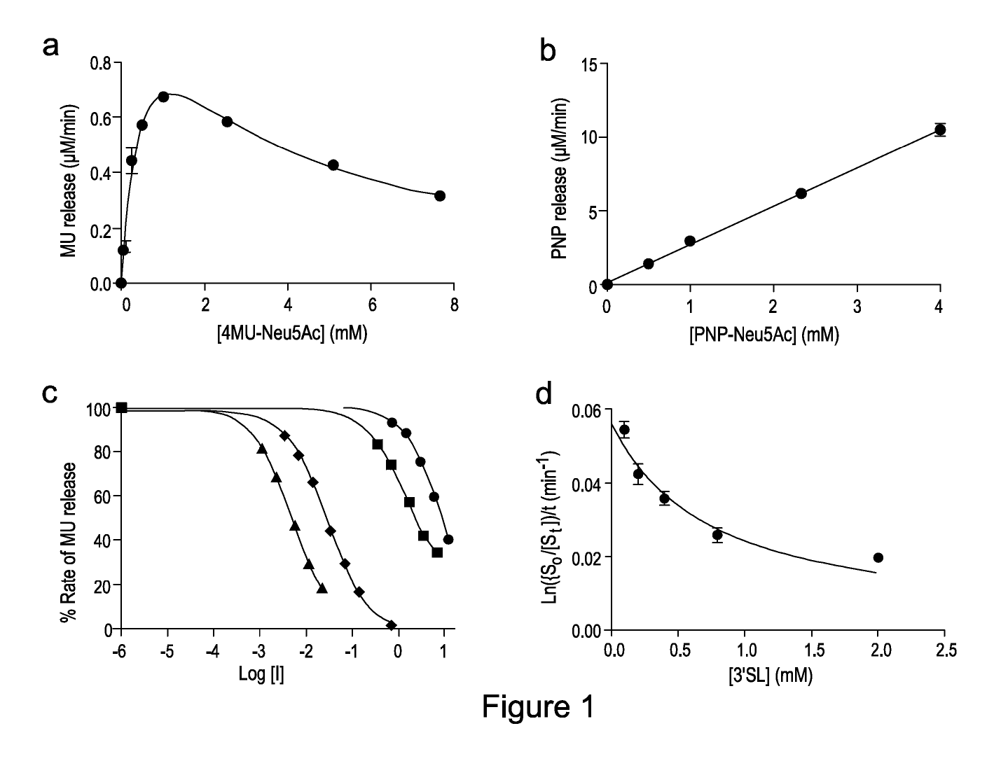

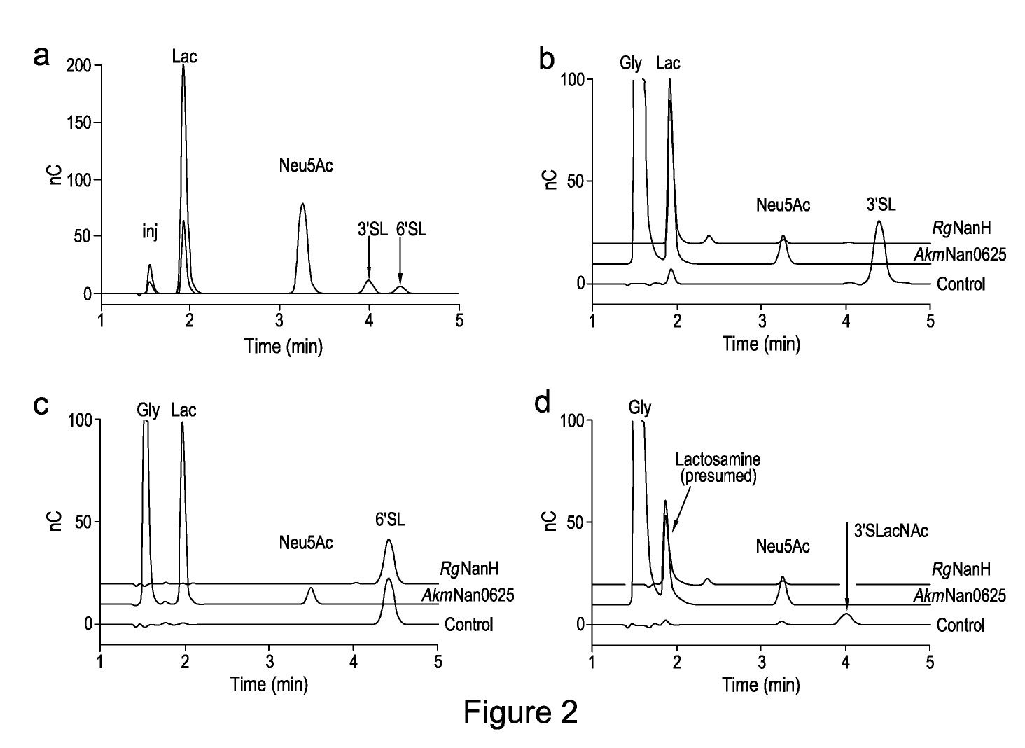

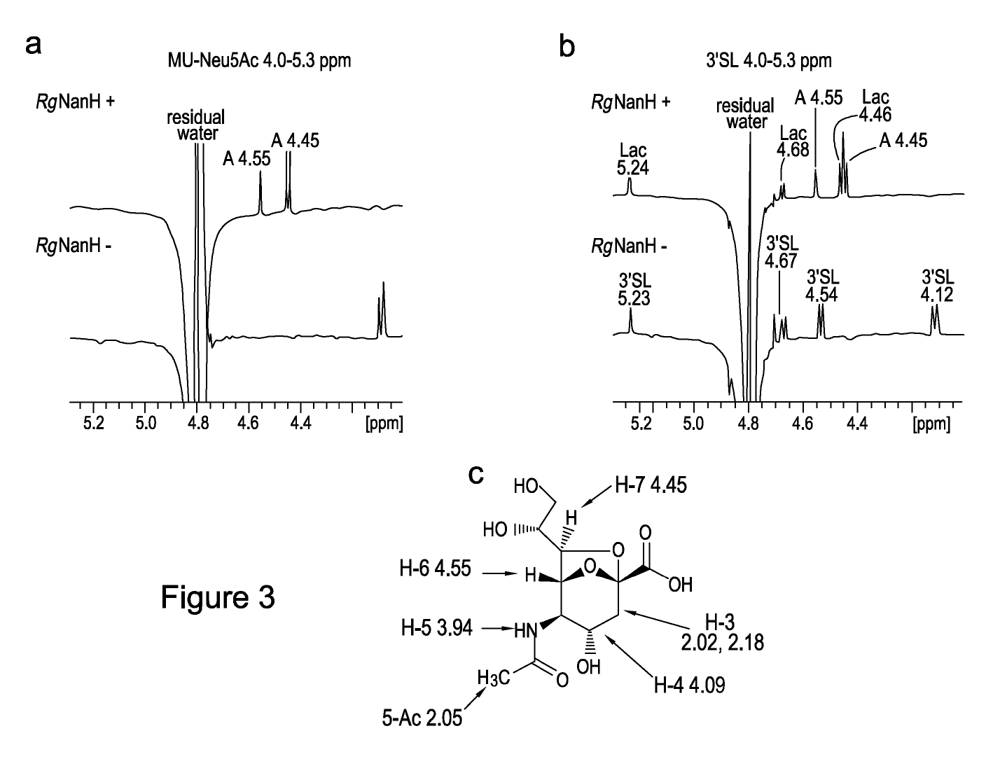

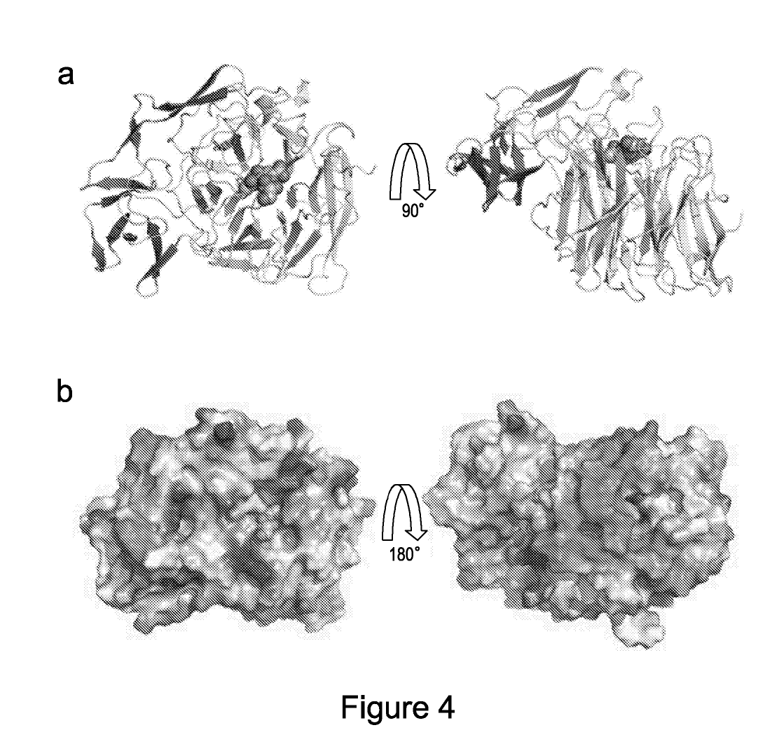

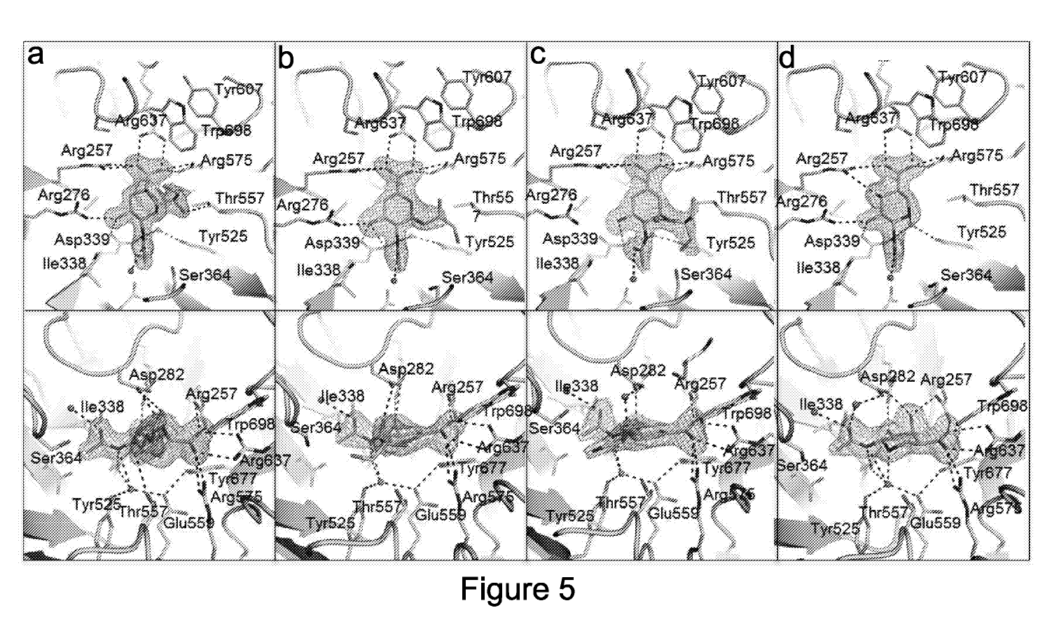

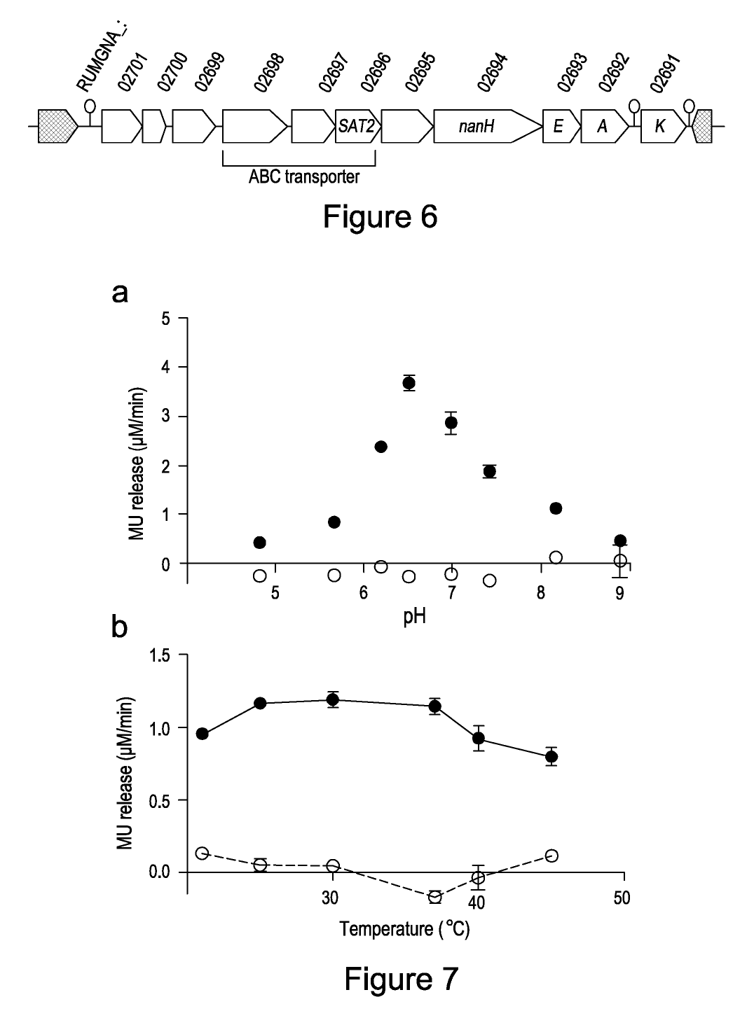

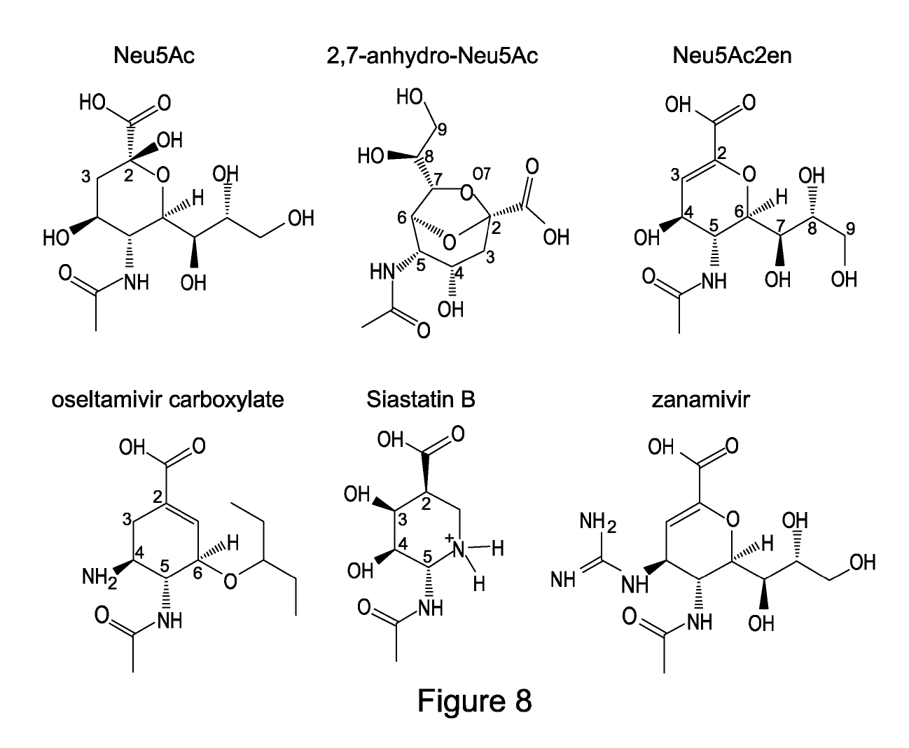

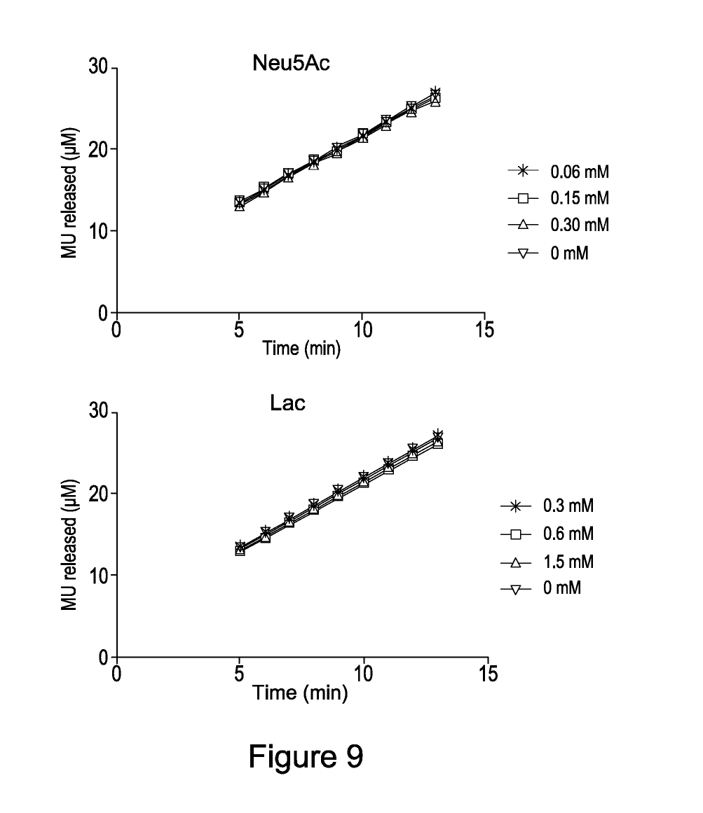

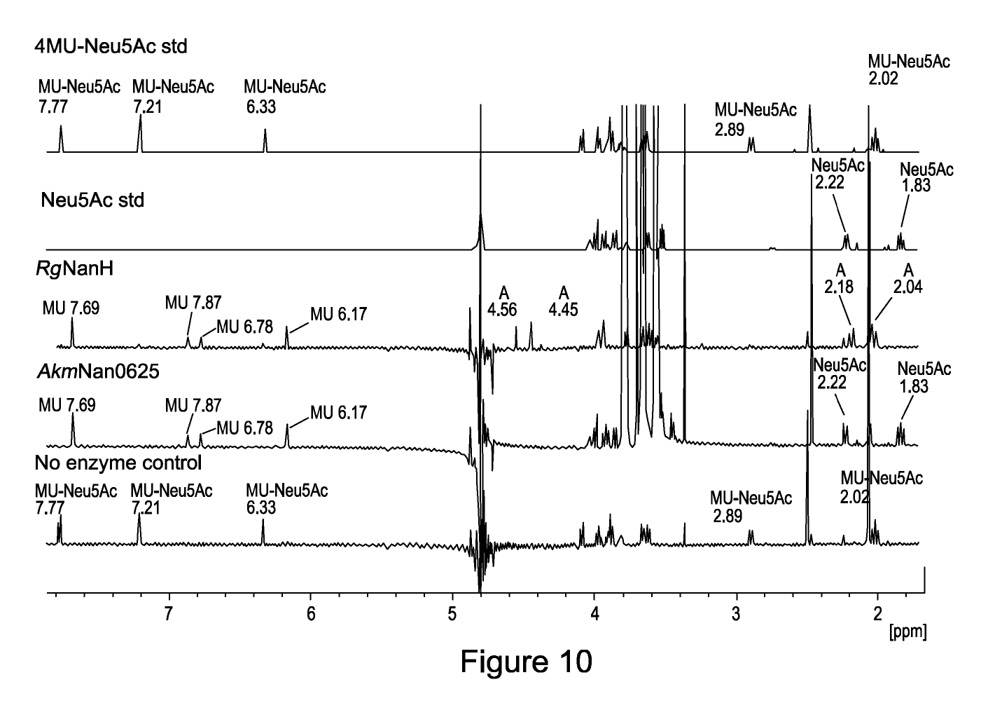

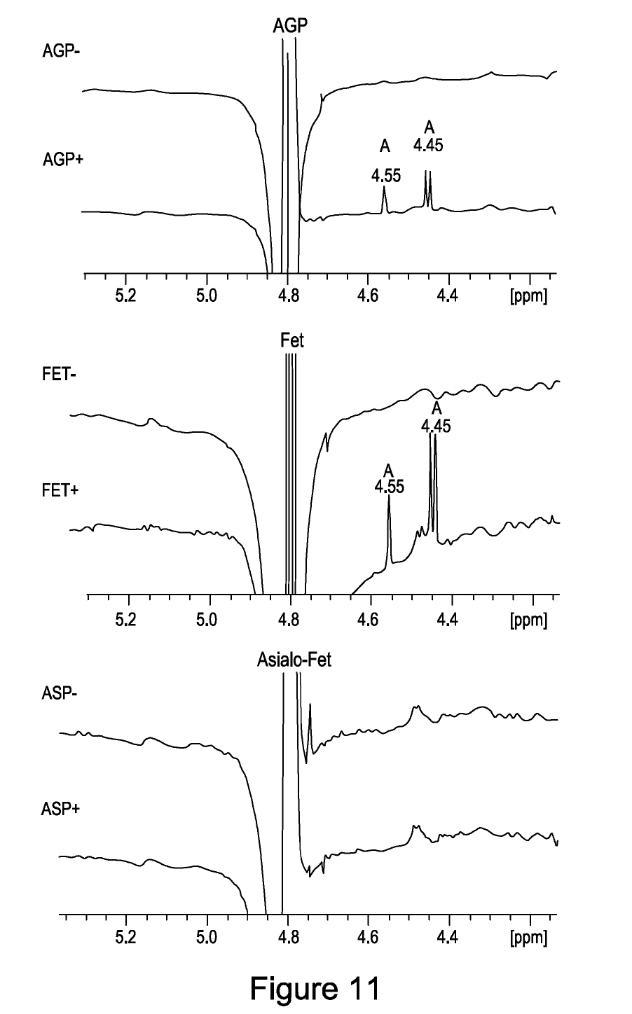

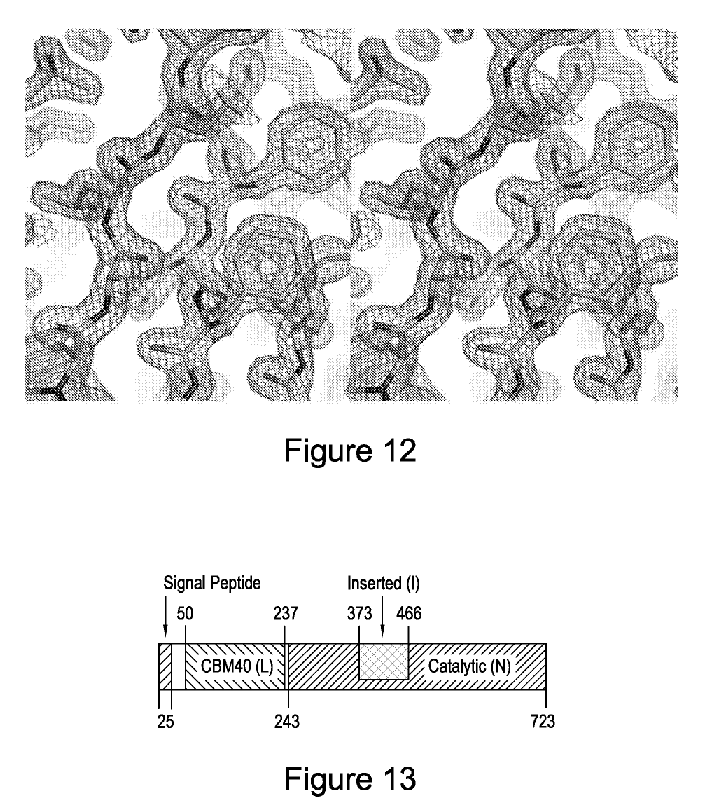

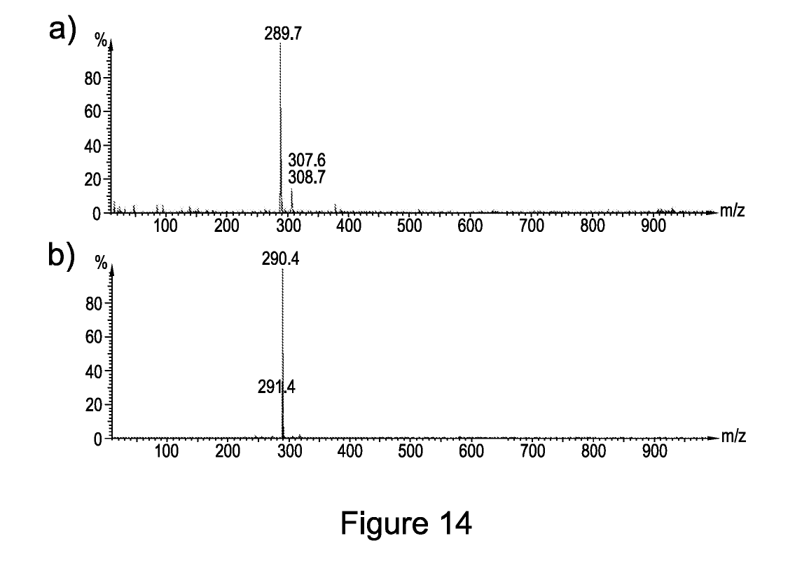

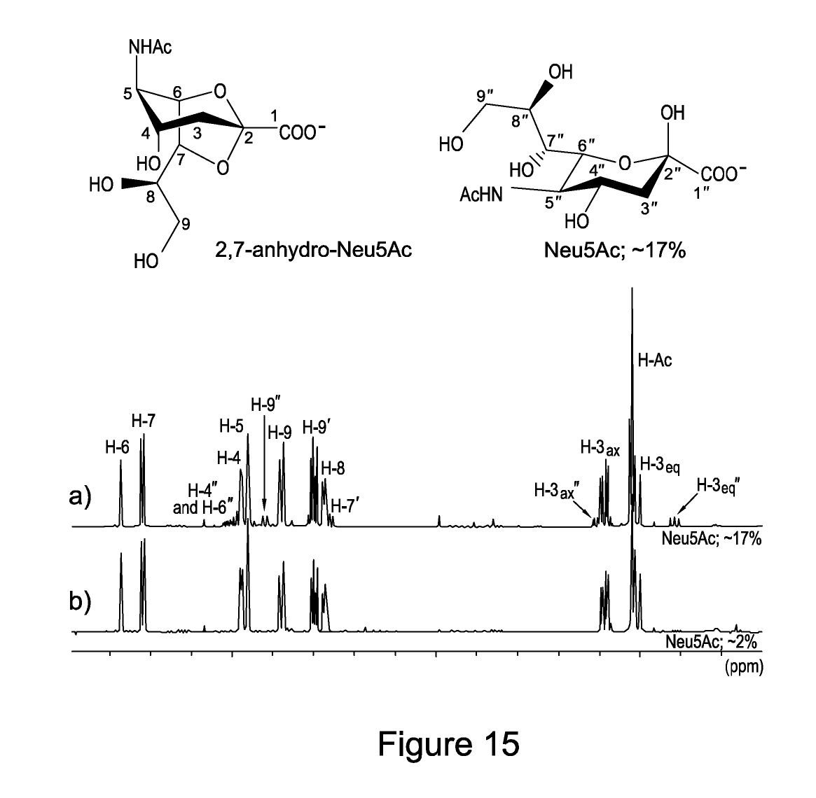

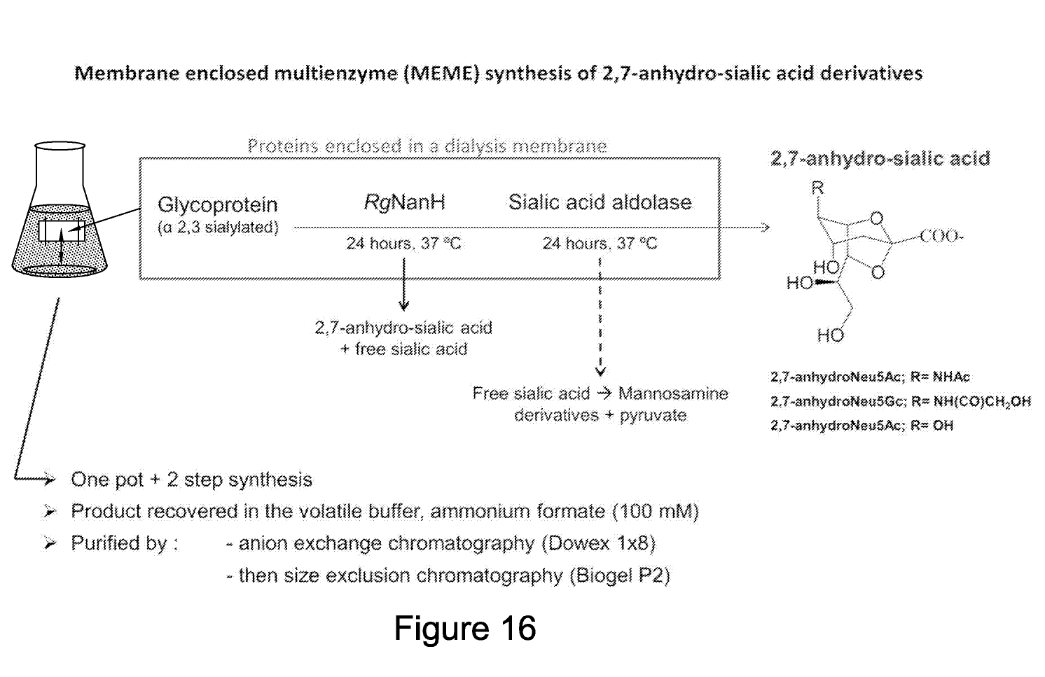

In order to determine the substrate specificity of R. gnavus ATCC 29149 sialidase (RgNanH), the full-length gene was heterologously expressed in Escherichia coli and the recombinant enzyme purified to homogeneity. The enzymatic activity of the recombinant protein was first assessed using the synthetic fluorescent substrate, 2′-(4-Methylumbelliferyl)-α-D-N-acetylneuraminic acid (4MU-Neu5Ac) (Table 1). RgNanH displayed a pH optimum of 6.5 and a broad temperature optimum ranging from 25° C. to 40° C. (FIG. 7), consistent with the environment of the human gut. These conditions (pH 6.5, 37° C.) were used to determine the kinetic parameters of RgNanH against the synthetic substrates 4MU-Neu5Ac and 2-O-(p-Nitrophenyl)-α-D-N-acetylneuraminic acid (PNP-Neu5Ac) (FIGS. 1a and b, Table 1). RgNanH showed 6.21×10³ min⁻¹ k_{cat} and 0.59 mM K_{M} against 4MU-Neu5Ac but a high K_{M} and low activity against PNP-Neu5Ac.

The activity of RgNanH against 4MU-Neu5Ac was further tested in the presence of a range of known neuraminidase inhibitors (FIG. 8). RgNanH showed moderate inhibition by 2-deoxy-2, 3-didehydro-D-N-acetylneuraminic acid (Neu5Ac2en) (IC₅₀ 1.4 mM) and a lower inhibition by its derivative 4-guanidino-Neu5Ac2en (zanamivir) (IC₅₀ 11.89 mM). Oseltamivir carboxylate (OC) was substantially more effective with an IC₅₀ of 30 pM (FIG. 1c, Table 2). Siastatin B was the best inhibitor of RgNanH with an IC₅₀ of 5 pM (FIG. 1c, Table 2). RgNanH was not inhibited by Lactose (Lac) or Neu5Ac (FIG. 9).

Preference of RgNanH in cleaving either α2-3 or α2-6 linked sialic acid was assessed by incubation of the enzyme with 3′SL and 6′-sialyllactose (6′SL), which contain α2-3 and α2-6 linked sialic acid, respectively (FIG. 1d, Table 3). The enzyme showed a K_{M} for 3′SL below detection limit, indicative of a very high affinity of the enzyme for this substrate and an estimated k_{cat} of 25.7 min⁻¹ whereas no enzymatic activity was observed in presence of 6′SL, demonstrating exclusive substrate specificity of the enzyme for α2-3 linkages. The higher activity (k_{cat}) of the enzyme for 4MU-Neu5Ac compared to 3′SL may be due to MU acting as a better leaving group. The enzyme showed substrate inhibition with both synthetic and natural substrates (FIGS. 1a and d, Table 1), with a lower Kᵢ for the 3′SL (0.76 mM), compared to 4MU-Neu5Ac (2.37 mM). In contrast, two sialidases AkmNan0625 and AkmNan1835 from Akkermansia muciniphila, used as controls, were active against both 3′SL and 6′SL (FIGS. 1a and b). The substrate and product specificity of the enzymes was further monitored by HPAEC-PAD (FIG. 2, Table 3) using substrates ranging from the monosaccharide galactose to the branched trisaccharide Lewis X conjugated to sialic acid with either α2-3 or α2-6 linkages. While the A. muciniphila sialidases released Neu5Ac from all substrates tested, RgNanH activity was only observed for α2-3 linked sialyl-oligosaccharides, as observed by the disappearance of the substrate peak and the appearance of a lower molecular weight peak that may correspond to the desialylated substrate. However no free Neu5Ac was observed from the action of RgNanH.

The products of the reaction were thus further monitored by ¹H NMR. The spectra clearly showed the presence of two ¹H NMR signals at 4.56 and 4.45 ppm (FIG. 3). These peaks are characteristic of 2,7-anhydro-α-N-acetylneuraminic acid (2,7-anhydro-Neu5Ac) and arise from the protons in positions 6 and 7 (FIG. 3a). Peaks corresponding to 2,7-anhydro-Neu5Ac were observed when RgNanH was incubated in presence of 4MU-Neu5Ac (FIG. 3a) or 3′SL (FIG. 3b). No changes in the peaks corresponding to 2,7-anhydro-Neu5Ac were observed after incubation of RgNanH with sialylated substrates for up to 24 h at 37° C. and no signal corresponding to free sialic acid could be detected, as also observed with the HPAEC-PAD experiments, indicating no spontaneous conversion of 2,7-anhydro-Neu5Ac to Neu5Ac. Conversely, there was no evidence of spontaneous conversion of Neu5Ac to 2,7-anhydro-Neu5Ac by NMR under the experimental conditions tested. The signals of 2,7-anhydro-Neu5Ac and their chemical shifts are shown in Table 6. This product was absent in control experiments using A. muciniphila sialidases (AkmNan0625 and AkmNan1835) or in absence of enzyme, confirming the specificity of the enzymatic reaction (FIG. 10). These data indicate that RgNanH produces 2,7-anhydro-Neu5Ac rather than Neu5Ac from sialylated oligosaccharides.

The ability of RgNanH to hydrolyse sialic acid from glycoproteins was investigated by incubation of the enzyme with human α-1-acid glycoprotein (AGP) and fetuin (Fet) both of which are known to contain α2-3 sialyl linkages, while asialo-fetuin was also included as a control. RgNanH showed activity against AGP and Fet, as monitored by ¹H NMR with the detection of peaks corresponding to 2,7-anhydro-Neu5Ac but not Neu5Ac, whereas no change was observed in the control reaction with asialo-fetuin in presence or absence of RgNanH (FIG. 11).

Taken together, these data demonstrate that RgNanH encodes an IT-sialidase, producing 2,7-anhydro-Neu5Ac selectively from α2-3 linked sialic acid substrates.

RgNanH Shares Structural Homology with IT-Sialidases

RgNanH sequence encodes a three domain modular protein with an N-terminal lectin-like domain (L-domain) classified as a member of the carbohydrate-binding module family 40 (CBM40, www.cazy.org), a catalytic domain (N-domain) classified as a member of the glycoside hydrolase family 33 (GH33; www.cazy.org), and a domain inserted into the catalytic domain (I-domain) (see FIG. 13). To date only two enzymes with IT-sialidase activity have been reported, NanL from Macrobdella decora (North American leech) (Chou et al. (1996)) and NanB from the human pathogen Streptococcus pneumoniae (Gut et al. (2008)). RgNanH shares ˜75% and ˜42% sequence identity over the whole protein length with NanL and NanB, respectively, and the same multidomain architecture. Homology is greatest in the catalytic domain (excluding the I-domain), showing ˜81% and ˜46% identity with NanL and NanB respectively. The CBM40 L-domain shares ˜67% and ˜30% identity and the I-domain shares 64% and 43%, both respective to NanL and NanB. The presence of CBM40 (SEQ ID NO: 5) in RgNanH may help i) target the IT-sialidase expressing bacteria to specific regions of the mucosal surface, ii) mediate adherence of the bacterium to host cells, iii) increase the catalytic efficiency of the IT-sialidase by increasing the concentration of enzyme in close proximity to its substrate.

In order to investigate the structural basis for the IT-sialidase reaction by RgNanH, we determined the crystal structure of RgNanH catalytic domain, free and in complex with the ligands identified above. RgNanH catalytic domain (residues 243-723) was heterologously expressed and purified, and the crystal structure of the N-domain with the inserted I-domain (NI-domain) was solved to a maximum resolution of 1.71 Å (Table 4). As shown in FIG. 4a, the N-domain adopts the canonical sialidase six-bladed β-propeller fold, which is shared with NanL (Luo et al. (1998)) (PDB 2SLI) and NanB (Xu, G. G. et al. Crystal Structure of the NanB Sialidase from Streptococcus pneumoniae. J Mol Biol 384, 436-449 (2008)) (PDB 2VW0). The N-domain contains four asparagine boxes, one in each of the first four β-propeller blades. These are common structural motifs in bacterial sialidases and form β-hairpin loops between β-strands three and four of a β-propeller blade, providing blade-to-blade interactions (Quistgaard, E. M. & Thirup, S. S. Sequence and structural analysis of the Asp-box motif and Asp-box beta-propellers; a widespread propeller-type characteristic of the Vps10 domain family and several glycoside hydrolase families. BMC structural biology 9, 46 (2009)). There is an extended β-hairpin loop in the equivalent position in blade five. The I-domain is formed by a loop extended from between two blades of the propeller and is primarily comprised of β-strands (FIG. 4a). The enzyme surface displays a significant charge bias, with the active site face being generally positive and the opposite face substantially negative (FIG. 4b). Soaking of the NI-domain crystals with 3′SL resulted in substrate turnover, leading to a protein ligand complex with 2,7-anhydro-Neu5Ac bound into the active site (FIG. 5). RgNanH active site presents the classical features of bacterial sialidases, shared by hydrolytic and trans-sialidases (FIG. 5a). These include: an arginine triad formed by Arg257, Arg637, and Arg575; a catalytic pair formed by Glu559 and Tyr677; a general acid base, Asp282; and a pocket to accommodate the N-acetyl group. As shown in FIG. 5a, the arginine triad forms electrostatic interactions with the 2,7-anhydro-Neu5Ac carboxylate group, orientating the ligand in the active site. Asp282 forms hydrogen bonds with the newly formed ethylene glycol group. The ligand N-acetyl group sits in a pocket between Ser364 and Ile338 with the amine providing hydrogen-bonding interactions to Asp339, and Glu559 and Tyr525 via a buried water molecule. The carbonyl group of the N-acetyl functional group may also interact with Asp282 via a water molecule.

The formation of the intramolecular sialosyl linkage specific to IT-sialidases has been proposed to be due to the positioning of a threonine underneath the glycerol group of the sialic acid substrate, forcing it into an axial position from which it can attack the ring carbon C2 (Luo, Y., Li, S. C., Li, Y. T. & Luo, M. The 1.8 angstrom structures of leech intramolecular trans-sialidase complexes: Evidence of its enzymatic mechanism. J Mol Biol 285, 323-332 (1999)). In RgNanH, this mechanism would appear to be conserved through Thr557. Furthermore, there is a hydrophobic stack formed by Tyr607 and Trp698 in front of the active site (FIG. 5a). This feature is likely to be responsible for the specificity of IT-sialidases for α2-3 linked substrates and also for creating a generally hydrophobic region of the active site, favouring nucleophilic attack by the glycerol group rather than an incoming water molecule (Luo et al. (1999)).

To gain further insights into the inhibition profile of this enzyme, crystal structures of RgNanH in complex with inhibitors tested enzymatically were investigated. Complexes with Neu5Ac2en, OC, and Siastatin B, bound in the active site were solved to 2.00 Å, 2.01 Å and 1.94 Å, respectively (Table 4; FIG. 5b, c, d; FIG. 12). It was not possible to obtain a crystal structure of zanamivir in complex with the enzyme, probably due to its low affinity for RgNanH (IC₅₀ of ˜12 mM). Neu5Ac2en (IC₅₀ of 1.4 mM) bound to RgNanH active site in a half-boat conformation, planar around the C2 (FIG. 5b). Most protein-inhibitor interactions were similar to those observed with 2,7-anhydro-NeuSAc, including the carboxylic acid group to the arginine triad, hydroxyl O4 to Arg276 and Asp339, and those made by the N-acetyl group. There is a hydrogen bond between the first hydroxyl of the glycerol group (O7) and Asp282 (FIG. 5b), which is similar to the interaction observed between Asp282 and the ethylene glycol group of 2,7-anhydro-NeuSAc. OC (IC₅₀ of ˜30 pM) is a substantially more potent inhibitor of RgNanH than Neu5Ac2en. Based on a cyclohexene scaffold rather than didehydropyran, OC has an amine at position C4 in comparison to the hydroxyl of Neu5Ac2en and a pentyl ether group in place of the glycerol group (FIG. 8). The C4 amine formed hydrogen bonds with RgNanH Asp282 and Asp339 (FIG. 5c). OC is shifted out of the active site by ˜0.6 Å, as compared to Neu5Ac2en, which may be due to repulsive effects between the inhibitor amino group and Arg276. The ether oxygen of the pentyl ether functional group may interact with Asp282 via a water molecule.

Siastatin B (IC₅₀ of ˜5 pM) was the most potent inhibitor tested. Siastatin B is based on an iminosugar scaffold with carboxylic acid, hydroxyl and N-acetyl functional groups at equivalent positions to NeuSAc, an additional hydroxyl group is present at position 3 (FIG. 8). The Siastatin B inhibitor bound to the active site in a chair conformation (FIG. 5d). Like Neu5Ac2en, the carboxylic acid group of Siastatin B interacts with the arginine triad, and the O4 hydroxyl with Arg276 and Asp339. Furthermore, the axial C3 hydroxyl of Siastatin B formed hydrogen bonds to Asp282 and Arg257 of the arginine triad. Asp282 may interact with the carbonyl of the ligand N-acetyl group via a water molecule. This was also observed in the 2,7-anhydro-Neu5Ac complex. Both the nitrogen of the carbohydrate ring and of the N-acetyl group formed hydrogen bonds to a buried water molecule, resulting in a more extensive hydrogen bonding network in this region than seen in the Neu5Ac2en and OC complexes.

IT-Sialidases are Overrepresented in IBD Gut Metagenomes

In order to assess the prevalence of IT-sialidase-positive bacteria across the human gut microbiota, initial assemblies of sequenced metagenomes of human stool samples from 99 healthy subjects and 25 IBD patients (Qin et al. (2010) were examined for the presence of IT-sialidases. We used Hidden Markov Models (pHMMs) to search the protein sequences resulting from translating the assembled coding sequences (14.1 million in all, with a mean length of 637 b.p.). All subjects' metagenomes were positive for a sialidase gene (mean 30 coding sequences per subject). The IT-sialidase was more scarcely detected, being present in 10.5% of all subjects, with a mean of 1.5 sequences per positive subject. However, of the non-IBD subjects, 8.1% were positive for IT-sialidase in contrast to 20% of the smaller group of IBD subjects. In the two groups respectively 1 in 830,000, and 1 in 422,000 of all the coding sequences were identified as IT-sialidase (Table 5). These data are consistent with an over-representation of IT-sialidases in IBD patients.

To further investigate the nature of the bacteria species possessing the IT-sialidase-encoding gene (RgNanH), in addition to the known Nan clusters, we performed a bioinformatics analysis using the same pHMMs (see Methods for details). We found that the genomes of 488 bacterial strains (6% of the 8,126 analyzed) representing 44 species, had IT-sialidase hits, of which 94% (457 strains), representing 32 species, tested positive for potential NanA, NanK, NanE1-encoding genes within a cluster spanning up to 15 contiguous gene loci. These strains represent 18% of all genomes with this cluster. A further six IT-sialidase positive species (all with genomes in draft form) matched these 3 genes but not in a cluster. Four of the IT-sialidase species were positive for homologues of the NanL, NanE2, RokA components of the B. fragilis pathway (in total, 1,796 strains possessed these components). All strains positive for the B. fragilis components lacked a match to NanE1 (Table 7). A total of 3,427 strains were found positive for either the NanL/RokA/NanE2 set of genes or the NanA/K/E1 cluster or both (if the latter genes are permitted to be present but not necessarily in a cluster then this rises to 4,127). Of these, 460 (13%) have an IT-sialidase gene. The CBM40 domain was associated with 75% of the IT-sialidases. Interestingly the IT-sialidase positive strains from species known to occur in the human gut seemed restricted to Firmicutes, in particular Clostridiales and Lactobacillales (see Table 7).

### DISCUSSION

Sialidases are a large group of enzymes which catalyse the cleavage of terminal sialic acids from complex carbohydrates on glycoproteins or glycolipids. Based on their substrate specificity and catalytic mechanism, sialidases can be separated into three different classes. Hydrolytic-sialidases cleave the glycosidic bond of terminal sialic acids and release free sialic acid, whereas trans-sialidases transfer the cleaved sialic acid to other glycoconjugates; according to the Enzyme Commission both classes belong to exo-α-sialidases (EC 3.2.1.18). Hydrolytic-sialidases usually have wide substrate specificity and cleave α2-3-, α2-6-, and α2-8-linked terminal sialic acids while trans-sialidases have a preference for α2-3-linked substrates. The third class is the IT-sialidase (EC 4.2.2.15) that is strictly specific for α2-3-linked sialic acids and produces 2,7-anhydro-Neu5Ac27. To date, only NanL from M. decora and NanB from S. pneumoniae have been assigned to this third class (Chou, M. Y., Li, S. C. & Li, Y. T. Cloning and expression of sialidase L, a NeuAc alpha 2→3Gal-specific sialidase from the leech, Macrobdella decora. J Biol Chem 271, 19219-19224 (1996); Gut, H., King, S. J. & Walsh, M. A. Structural and functional studies of Streptococcus pneumoniae neuraminidase B: An intramolecular trans-sialidase. Febs Lett 582, 3348-3352 (2008)). These enzymes are unique in that they catalyse an IT-reaction in which the 07-hydroxyl group of the bound sialic acid glycerol group attacks the positively charged C2 atom of the oxocarbenium intermediate. This altered reaction pathway leads to release of 2,7-anhydro-Neu5Ac instead of Neu5Ac, the reaction product for hydrolytic sialidases. In the present application, it is shown that RgNanH is a novel member of this class, the first one identified and characterised in members of the human gut microbiota. The enzyme showed strict specificity towards α2-3 glycosidic substrate linkages as tested by HPAEC and ¹H NMR using a range of natural substrates from disaccharides to branched tetrasaccharides. The production of 2,7-anhydro-Neu5Ac in the reaction with 3′SL and synthetic substrates 4MU-Neu5Ac and PNP-Neu5Ac confirmed that RgNanH is an IT-sialidase. Furthermore human α-1-acid glycoprotein (AGP) and fetuin (Fet) were both substrates of RgNanH, in agreement with the presence of α2-3 sialic acid linkages in these proteins; Neu5Ac-α2-6-Gal, Neu5Ac-α2-3-Gal as well as Lewis X epitopes are found in AGP (Taguchi, K., Nishi, K., Chuang, V. T. G., Maruyama, T. & Otagiri, M. Molecular Aspects of Human Alpha-1 Acid Glycoprotein—Structure and Function. (2013)), whereas Fet also contains sialylated α2-6-Gal and α2-3-Gal epitopes (Baenziger, J. U. & Fiete, D. Structure of the Complex Oligosaccharides of Fetuin. J Biol Chem 254, 789-795 (1979)). The RgNanH crystal structures showed active site features associated with the IT-sialidase class. Of particular importance is the conservation of Thr557, which seems to sterically force the substrate glycerol group into a position from where it can attack the C2 atom, and of the Tyr607 and Trp698 hydrophobic stack, proposed to be responsible for the observed substrate specificity and also the maintenance of a dry active site promoting nucleophilic attack by the glycerol group (Luo, Y., Li, S. C., Chou, M. Y., Li, Y. T. & Luo, M. The crystal structure of an intramolecular trans-sialidase with a NeuAc alpha 2→3Gal specificity. Struct Fold Des 6, 521-530 (1998)).

RgNanH displayed activity against all α2-3 linked substrate tested. The active site has been considered to be the pocket accommodating the terminal sialic acid residue of the substrate. However the reaction rate is greatly influenced by the glycone moiety, as shown by the variation in K_{M} and efficiency of cleavage (k_{cat}/K_{M}) between 4M U-Neu5Ac and PNP-Neu5Ac even though both fluorophore and chromophore have similar leaving abilities (pKₐ 7.79 and 7.23, respectively). 3′SL also displays much tighter apparent affinity than the synthetic substrates. Similar results have also been observed in other sialidases. For example, NanC, an α2-3-linkage specific sialidase from S. pneumoniae that shares 42% identity with RgNanH, shows a preference for LacNAc-compared with Lac-based substrates, and demonstrates reduced activity towards fucosylated glycans (Parker, R. B., McCombs, J. E. & Kohler, J. J. Sialidase Specificity Determined by Chemoselective Modification of Complex Sialylated Glycans. Acs Chem Biol 7, 1509-1514 (2012)). These data suggest that sialidases such as RgNanH have adapted to complex substrates and that enzyme substrate interactions are not limited to the terminal sugar residue. Furthermore both 3′SL and 4MU-Neu5Ac showed substrate inhibition, suggesting a potential mechanism for regulation of this enzyme activity.

Siastatin B was the most effective inhibitor of RgNanH with an IC₅₀ of ˜5 pM, compared to zanamivir (IC₅₀ of −12 mM), Neu5Ac2en (IC₅₀ of 1.4 mM) and OC (IC₅₀ of 30 pM). Our crystal structures of RgNanH inhibitor complexes provide a structural explanation for the differences in inhibition between the chemical inhibitors tested. Neu5Ac2en, an analogue of Neu5Ac dehydrated at C2, is a general sialidase inhibitor that inhibits both viral and bacterial sialidases (Gut, H., Xu, G. G., Taylor, G. L. & Walsh, M. A. Structural Basis for Streptococcus pneumoniae NanA Inhibition by Influenza Antivirals Zanamivir and Oseltamivir Carboxylate. J Mol Biol 409, 496-503 (2011); Vonitzstein, M. et al. Rational Design of Potent Sialidase-Based Inhibitors of Influenza-Virus Replication. Nature 363, 418-423 (1993)), including the hydrolytic sialidase expressed by Bacteroides thetaiotaomicron (Park, K. H. et al. Structural and biochemical characterization of the broad substrate specificity of Bacteroides thetaiotaomicron commensal sialidase. Bba-Proteins Proteom 1834, 1510-1519 (2013)). It has been proposed that Neu5Ac2en is a poor inhibitor and transition state mimic of IT-sialidases because the glycerol group is constrained by the C2=C3 bond and unable to occupy the axial position required for catalysis (Luo et al. (1998)), as also seen in the RgNanH-Neu5Ac2en complex (FIG. 5b). OC and zanamivir are variants of Neu5Ac2en originally designed to improve inhibition of the viral neuraminidase from influenza. In the case of OC, these modifications led to an improvement in inhibition of RgNanH by ˜50 fold (IC₅₀: 1.4 mM to 30 pM). This is likely due to enhanced interactions from the C4 amine group, as seen in the complex structure (FIG. 5c). In contrast, Neu5Ac2en makes a direct hydrogen bond to Asp282 via the hydroxyl O7. This interaction is not present in the complex with OC, although there may be a water-mediated interaction from Asp282 to the ether oxygen. In viral neuraminidases, and hydrolytic sialidases such as NanA from S. pneumoniae, there are substantial hydrophobic interactions with the pentyl ether functional group of OC (Gut, et al. (2011); Vonitzstein, et al. (1993)); these interactions are not present in RgNanH, which may explain why OC is less potent towards this IT-sialidase. Zanamivir was a very poor inhibitor of RgNanH, which is not surprising since the inhibitor has a much bulkier guanidino substitution at the C4 position, which based on the above complex crystal structures, would clash with Arg276 and Asp339. The complex between siastatin B and RgNanH provides a structural explanation for its effective inhibition of the enzyme (FIG. 5d). Siastatin B adopts a chair conformation rather than the more planar, half-boat Neu5Ac2en conformation. Furthermore, in comparison to Neu5Ac2en and OC, siastatin B makes a more extensive hydrogen-bonding network underneath the ligand via the buried water. Siastatin B also makes extensive interactions through the O3 hydroxyl to Asp282 and Arg257. Finally, although both Neu5Ac2en and siastatin B form hydrogen bonds to Asp282 (Neu5Ac2en via the glycerol group), siastatin B achieves such interaction without paying the entropic penalty required to avoid a steric clash between the glycerol group and Thr557. Overall the inhibitor data showed considerable differences between RgNanH and viral neuraminidases.

Biochemical assays highlighted differences in sialylated substrate specificity between R. gnavus and other gut mucin-degrading bacteria, such as A. muciniphila which is a dedicated mucus utiliser that has found to increase intestinal permeability in mice (Belzer, C. & de Vos, W. M. Microbes inside-from diversity to function: the case of Akkermansia. Isme J 6, 1449-1458 (2012); Everard, A. et al. Cross-talk between Akkermansia muciniphila and intestinal epithelium controls diet-induced obesity. Proc Natl Aca Sci USA 110 (2013)). Many enteric commensal and pathogenic bacteria can utilise sialic acids from their hosts owing to the presence of a Nan cluster (Almagro-Moreno, S. & Boyd, E. F. Insights into the evolution of sialic acid catabolism among bacteria. BMC Evol Biol 9, 118 (2009); Yu, Z. T., Chen, C. & Newburg, D. S. Utilization of major fucosylated and sialylated human milk oligosaccharides by isolated human gut microbes. Glycobiology 23, 1281-1292 (2013)), but not all have the ability to release sialic acid from glycoproteins. Among gastrointestinal commensals, Bacteroidetes species are found at high abundance and many of them express sialidases in culture (Moncla, B. J., Braham, P. & Hillier, S. L. Sialidase (Neuraminidase) Activity among Gram-Negative Anaerobic and Capnophilic Bacteria. J Clin Microbiol 28, 422-425 (1990)). However, some bacteria, such as B. thetaiotaomicron encode the sialidase required to cleave and release this terminal sugar from the mucosal glycoconjugates, but do not encode a sialic acid lyase/aldolase homologue and lack the catabolic pathway (that is, the Nan clusters) required to consume the liberated monosaccharide. Presumably, the release of sialic acids allows B. thetaiotaomicron to access highly coveted underlying carbohydrates in the mucus (Koropatkin, N. M., Cameron, E. A. & Martens, E. C. How glycan metabolism shapes the human gut microbiota. Nature reviews. Microbiology 10, 323-335 (2012); Marcobal, A., Southwick, A. M., Earle, K. A. & Sonnenburg, J. L. A refined palate: Bacterial consumption of host glycans in the gut. Glycobiology 23, 1038-1046 (2013)). B. fragilis or E. coli on the other hand possesses the complete pathway of sialic acid catabolism including the hydrolytic sialidase gene (Brigham et al. (2009); Nakayama-lmaohji, H. et al. Characterization of a gene cluster for sialoglycoconjugate utilization in Bacteroides fragilis. J Medl Invest 59, 79-94 (2012)). Both B. fragilis and E. coli catabolic genes are upregulated in response to available free sialic acid (Nakayamadmaohji, H. et al. Characterization of a gene cluster for sialoglycoconjugate utilization in Bacteroides fragilis. The journal of medical investigation: JMI 59, 79-94 (2012)). A recent study reported that mice monoassociated with B. thetaiotaomicron exhibited a significantly higher concentration of free Neu5Ac versus germ-free mice, consistent with the ability of B. thetaiotaomicron to liberate but not consume, the monosaccharide, whereas colonisation of mice with B. fragilis, which is able to catabolise Neu5Ac, did not result in increased free sialic acid (Ng, K. M. et al. Microbiota-liberated host sugars facilitate post-antibiotic expansion of enteric pathogens. Nature 502, 96-99 (2013)). This repartition of free sialic acid in the gut is important as some bacterial pathogens such as Salmonella typhimurium and Clostridium difficile, do not encode sialidases and thus rely on the sialic acid liberated by the resident microbiota to expand in the mucosal environment (Hoyer, L. L., Hamilton, A. C., Steenbergen, S. M. & Vimr, E. R. Cloning, sequencing and distribution of the Salmonella typhimurium LT2 sialidase gene, nanH, provides evidence for interspecies gene transfer. Mol Microbiol 6, 873-884 (1992); Sebaihia, M. et al. The multidrug-resistant human pathogen Clostridium difficile has a highly mobile, mosaic genome. Nat Genet 38, 779-786 (2006)). This cross-feeding activity has also been reported between members of Bifidobacteria, e.g. Bifidobacterium breve UCC2003 (containing a functional Nan cluster for sialic utilisation) can utilise sialic acid released by the sialidase activity of B. bifidum PRL2010 (Egan, M., O'Connell Motherway, M., Ventura, M. & van Sinderen, D. Metabolism of sialic acid by Bifidobacterium breve UCC2003. Appl Environ Microbiol 80, 4414-4426 (2014)).

R. gnavus ATCC 29149 is different to the above as it possesses the complete Nan cluster and an IT-sialidase producing 2,7-anhydro-Neu5Ac instead of Neu5Ac from α2-3 linked sialic acid substrates, consistent with the bacteria ability to grow on 3′SL but not sialic acid, Lac or 6′SL and the induced expression of the Nan cluster and RgNanH on 3′SL (Crost et al. (2013)). Bioinformatics analysis revealed that the presence of IT-sialidases is shared by other members of the gut microbiota, in particular Blautia hansenii and R. torques, all 10 strains of C. perfringens with available genome data, C. sp. 7 2 43 FAA, C. celatum, C. nexile, C. spiroforme, 3 unclassified Lachnospiraceae, more than 100 strains of S agalactiae, and 3 of the genome-sequenced publicly available Lactobacillus salivarius strains. Of 1,165 strains testing positive for a sialidase and the Nan clusters, 40% (457 strains) tested positive specifically for the IT-sialidase (the remainder matched the sialidase domain but lacked the I-domain). The detection of IT-sialidase homologues in at least 11% of gut metagenomes of a population of diseased and healthy humans is in line with this analysis, showing that this enzyme is widespread across gut bacteria, especially in Firmicute.

The specific niche colonisation of these bacteria may reflect an adaptation to particular mucus glycosylation profiles. The R. gnavus mucin-utilisation enzymatic profile, which is mainly based on the release and use of sialic acid to support growth (Crost et al. (2013)) appears particularly well adapted to digest mucin with short chains terminated by sialic acid, such as sialyl-Tn-antigen, which is found in higher proportion in IBD patients (Larsson, J. M. et al. Altered O-glycosylation profile of MUC2 mucin occurs in active ulcerative colitis and is associated with increased inflammation. Inflamm Bowel Dis 17, 2299-2307 (2011)). On the contrary, other mucin degraders such as A. muciniphila or B. thetaiotaomicron cannot utilise sialic acid as carbon source, so will be disadvantaged by the mucin glycosylation profile of IBD patients, while more adapted to utilise complex mucin glycan structures (Larsson, J. M. et al. A complex, but uniform O-glycosylation of the human MUC2 mucin from colonic biopsies analysed by nanoLC/MSn. Glycobiology 19, 756-766 (2009)) that require the synergistic action of several glycoside hydrolases (Tailford, L. E. et al. Mucin glycan foraging in the human gut microbiome. Front Genet 6, 81 (2015)). Furthermore, the IT-sialidase may provide gut microbes such as R. gnavus ATCC 29149 with an additional competitive nutritional advantage, allowing the bacteria to thrive within mucosal environments by scavenging sialic acid from host mucus in a form, 2,7-anhydro-Neu5Ac, which may not be readily accessible to other members of the gut microbiota, thus limiting loss to other microbiota residents and/or exploitation by enteric pathogens. This ‘selfish’ model of mucosal glycan utilisation can contribute to the disproportionate representation of R. gnavus reported in ulcerative colitis and Crohn's disease patients (Png, C. W. et al. (2010); Joossens, M. et al. Dysbiosis of the faecal microbiota in patients with Crohn's disease and their unaffected relatives. Gut 60, 631-637 (2011); Willing, B. P. et al. A pyrosequencing study in twins shows that gastrointestinalmicrobial profiles vary with inflammatory bowel disease phenotypes. Gastroenterology 1 39, 1844-1854 e1841 (2010)).

### Example 2

Enzymatic Synthesis of 2,7-Anhydro-Neu5Ac

Recombinant R. gnavus IT-sialidase, full-length (RgNanH (SEQ ID NO:1)) or catalytic domain (RgGH33 (SEQ ID NO:6)), is used for the enzymatic synthesis of 2,7-anhydro-Neu5Ac. The enzyme is produced in Escherichia coli and purified by immobilised metal anion chromatography and gel filtration on an Akta system (as described in Tailford et al. (2015)) and used for the enzymatic synthesis of 2,7-anhydro-Neu5Ac. This may be used in bacterial growth assays or to test potential applications of the compound. The RgNanH or RgGH33 so produced is used to enzymatically synthesise 2,7-anhydro-Neu5Ac using a modification of the established protocol in U.S. Pat. No. 5,312,747 from 4-Methylumbelliferyl-N-acetylneuraminic acid (4MU-Neu5Ac). 4MU-Neu5Ac (commercially available) is incubated with recombinant enzyme for 18 hours. The product of the reaction is then recovered following Folch partitioning and Bio-Gel chromatography and analysed by ¹H-NMR and mass spectrometry.

An alternative method has been developed to allow the large-scale production of 2,7-anhydro-Neu5Ac. Here the substrate used was fetuin, a glycoprotein containing ˜8% of α2,3 linked sialic acid in weight. The reaction medium (RgGH33 or RgNanH+Fetuin) was enclosed in a dialysis membrane (7K MWCO), which allows easy separation of the reaction product from the proteins. A volatile buffer was used, ammonium formate 100 mM, which was removed by freeze-drying the sample. The fetuin was previously dialysed in this buffer to remove the free sialic acid. The product was then purified by size exclusion chromatography using a Bio-Gel P2 column and analysed by ¹H-NMR and mass spectrometry.

In an optional additional step, following incubation of the substrate with the RgNanH or RgGH33 enzyme, a commercial sialic acid aldolase was added to the reaction mixture. The aldolase was used to convert free sialic acid into smaller and uncharged enzymatic products which were more easily eliminated during the anion exchange and size exclusion chromatography steps, producing a higher yield and greater purity of 2,7-anhydro-Neu5Ac.

Further details of the enzymatic production method are as follows:

### Materials

Fetuin from bovine serum, ammonium formate and Dowex 1×8 anion exchange resin were purchased from Sigma Aldrich (St Louis, USA). Sialic acid aldolase from Escherichia coli was from Carbosynth Limited (Compton, UK), BioGel P2 from Bio-Rad laboratories and SnakeSkin™ Dialysis Tubing, 7K MWCO, 22 mm from Thermo Fisher Scientific (Hemel Hempstead, UK). Nanopure water (18.2 MΩ·cm; NanoPure Thermo fisher Barnstead Water Reverse osmosis Purification System) was used for buffer preparation and for purification. RgNanH was produced and purified as described previously (Tailford L E, Owen C D, Walshaw J, Crost E H, Hardy-Goddard J, Le Gall G, de Vos W M, Taylor G L, Juge N. Discovery of intramolecular trans-sialidases in human gut microbiota suggests novel mechanisms of mucosal adaptation. Nat Commun. 2015 Jul. 8; 6:7624).

### Synthesis of 2,7-anhydro-sialic Acid

A dialysis membrane (7K MWCO) containing fetuin (0.3 mM) was incubated in 100 mM ammonium formate buffer pH 6.5 for 2 h at 37° C. under gentle shaking (115 rpm). Following the addition of 50 nM RgNanH (in Tris buffer 40 mM pH 7.9) into the dialysis membrane, the reaction mixture was further incubated in 100 mM ammonium formate buffer (pH 6.5) at 115 rpm, 37° C. for 24 h. Sialic acid aldolase (17.4 units in 100 mM ammonium formate) was added to the membrane enclosed reaction mixture. Following a further 20 h incubation at 115 rpm and 37° C., 100 mL of buffer was recovered, diluted with 100 mL of ultrapure water and freeze dried. The membrane enclosed reaction mixture was dialysed with 100 mL ultrapure water for 2 h and again overnight under 115 rpm shaking and at 37° C. The water was recovered, mixed with the dried synthesis buffer and freeze dried. After complete dryness, the powder was dissolved in 100 mL ultrapure water and freeze dried again 3 times to remove volatile salts.

### Purification of 2,7-anhydro-sialic Acid

The freeze-dried sample corresponding to crude 2,7-anhydro-Neu5Ac was dissolved in ultrapure water (2 mL) and purified by anion exchange chromatography using a Dowex 1×8 column. The anion exchange resin was first equilibrated with ultrapure water (200 mL) before applying the sample. After washing with ultrapure water (60 mL) and 0.001 M ammonium formate buffer (25 mL), 2,7-anhydro-Neu5Ac was eluted with 150 mL of ammonium formate buffer from 0.005 M to 0.05 M and freeze dried. After complete dryness, the powder was dissolved in ultrapure water (100 mL) and freeze dried again 3 times to remove volatile salts. 2,7-anhydro-Neu5Ac dissolved in ultrapure water (1.5 mL), centrifuged and filtered on a PTFE 0.45 μm membrane was then desalted by BioGel P2 size exclusion chromatography. The obtained 2,7-anhydro-Neu5Ac was collected and freeze dried.

### Analytical Methods

For electrospray ionisation mass spectrometry (ESI-MS) analysis, 2,7-anhydro-sialic acid was dissolved in methanol (0.1 mg/mL) and filtered on a PTFE 0.45 μm membrane.

MS spectra were acquired on Expression CMS^{L} (Advion, Ithaca, USA) with ESI ionisation using direct injection operated in a negative ion mode. Advion Data Express (version 2.2.29.2) software package was used to evaluate the MS data.

For Nuclear magnetic resonance (NMR) analysis, 2,7-anhydro-sialic acid (5 mg) was dissolved in D2O (800 μL) and 600 μL of this was transferred into a 5 mm o.d. NMR tube for spectral acquisition. NMR spectra were run on a 600 MHz Bruker Avance III HD spectrometer (Bruker Biospin GmbH, Rheinstetten, Germany) fitted with a TCI cryoprobe and running Topspin 3.2 software. All experiments were performed at 300 K and TSP was used as an internal standard. ¹H NMR spectra were acquired using the noesygpprld pulse sequence for suppression of the residual water signal. Spectra were acquired with 32678 complex points in the time domain, spectral width 20.5 ppm, acquisition time 2.67 s, relaxation delay 3.0 s and 64 scans. Fourier transformation was carried out with zero filling to 65536 points and exponential multiplication to give line broadening of 0.3 Hz. A proton decoupled ¹³C spectrum of the same solution was acquired at 151 MHz using the zgpg30 pulse sequence with 32678 complex time domain points, spectral width 220.9 ppm, acquisition time 0.98 s, relaxation delay 2 s and 7200 scans. Fourier transformation was carried out with zero filling to 65536 points and exponential multiplication to give 3.0 Hz line broadening. The spectra were referenced using an external standard of methanol (δ_{c}=51.43 ppm) in D2O. An HSQC spectrum of the same sample was recorded with the hsqcetgpprsisp2.2 pulse sequence with spectral widths of 12 ppm (¹H) and 165 ppm (¹³C), a 2048 (t2)×256 (t1) point acquisition matrix which was zero filled to 2048×1024 on 2D Fourier transformation, and 16 scans per t1 experiment. Standard two dimensional NMR techniques such as COSY and HSQC were performed for complete and unambiguous assignment of the proton and carbon signal. ¹³C signals were assigned using the HSQC spectrum based on the known ¹H chemical shifts.

### Results and Discussion

### Membrane Enclosed Enzymatic Synthesis of 2, 7-Anhydro-Neu5Ac

Here we used the recombinant IT-sialidase (RgNanH) from R. gnavus ATCC 29149 as described herein. and commercially available fetuin which contains about 8% in weight of a 2,3 linked sialic acid (Lee et al. (2002) Enzyme and Microbial Technology 31(6): 742-746Campo et al. (2007) Organic & Biomolecular Chemistry 5(16): 2645-2657) for the enzymatic biosynthesis of 2,7-anhydro-Neu5Ac. Fetuin was chosen as substrate as it is cheap and readily available. A Membrane Enclosed Enzymatic Catalysis (MEEC) (Bednarski et al. (1987) Journal of the American Chemical Society 109(4): 1283-1285) approach was used to achieve rapid and efficient recovery of the reaction product. This technique is based on the containment of the soluble enzyme in a dialysis membrane allowing the product to go through and be more readily recovered in the reaction buffer. Using this experimental approach, the enzyme (RgNanH) and its substrate (fetuin) were enclosed in the same dialysis membrane and therefore efficiently separated from the produced monosaccharide. The 2,7-anhydro-sialic acid derivative recovered in the reaction buffer was purified by anion exchange chromatography on Dowex 1×8 resin (chloride form) and size exclusion chromatography on a Bio-gel P-2 column in order to remove the salts. Electrospray ionisation mass spectroscopy (ESI-MS) and ¹H and ¹³C nuclear magnetic resonance (NMR) were used to monitor the purity of the reaction product. The mass spectrum was identical to that of 2,7-anhydro-Neu5Ac obtained by methanolysis of Neu5Ac (Lifely et al. Carbohydrate Research 107: 187-197 (1982)) or following enzymatic reaction of 4MU-Neu5Ac with the leech IT-sialidase (Li et al. J Biol Chem. 1990 Dec. 15; 265(35):21629-33) (FIG. 14 (a)). This process achieved 44% yield according to the 8% a 2,3 linked sialic acid. However the recovery product contained 85% 2,7-anhydro-Neu5Ac and 15% of free sialic acid (Neu5Ac), as determined by ¹H NMR (FIG. 15 (a)). No Neu5Ac was detected in a control reaction in absence of RgNanH, suggesting that no spontaneous degradation of fetuin occurred under the synthesis conditions or even after prolonged period up to 50 h dialysis, as monitored by ESI-MS (data not shown).

### Coupling Reaction of IT-Sialidase and Sialic Acid Aldolase

Considering the difficulty and the low efficiency of the separation of free Neu5Ac from 2,7-anhydro-Neu5Ac, a commercially available sialic acid aldolase from E. coli was introduced into the dialysis membrane with RgNanH and fetuin. Sialic acid aldolases are efficient biocatalysts that convert free Neu5Ac into N-acetyl-mannosamine (ManNAc) and pyruvate (Li et al. Appl Microbiol Biotechnol. 2008 July; 79(6):963-70). Since 2,7-anhydro-Neu5Ac is resistant to degradation by sialic acid aldolase, this enzyme was used to convert free sialic acid into smaller or uncharged enzymatic products which could be more easily eliminated during the anion exchange and size exclusion chromatography steps, as shown by MS (FIG. 14 (b)) and NMR (FIG. 15 (b)). The 2,7-anhydro-Neu5Ac obtained during this one pot Membrane enclosed multi-enzyme (MEME) synthesis was about 95% pure, with a Neu5Ac amount reduced to 2% (33% yield; 95% purity, 2% of Neu5Ac, 3% of protein residues). The multi-step enzymatic synthesis of 2,7-anhydro-Neu5Ac developed here is efficient, low cost and scalable. The MEME protocol for the synthesis of 2,7-anhydro-sialic acid derivatives is depicted in FIG. 16.

### Example 3

Impact of IT-Sialidase on Mucin Properties and Pathogen Adherence

Mucin is treated with IT-sialidase (ITS) and the ability of gut and respiratory pathogens to adhere and proliferate on ITS-treated mucin is tested using in vitro assays. Mucin samples from human cell lines are enzymatically treated with recombinant ITS. The release of 2,7-anhydroSA is monitored by ¹H-NMR (Crost, E. H. et al. (2013)). The removal of sialic acid is further confirmed by force spectroscopy (Gunning, A. P., Kirby, A. R., Fuell, C., Pin, C., Tailford, L. E., Juge, N. Mining the “glycocode”—exploring the spatial distribution of glycans in gastrointestinal mucin using force spectroscopy. FASEB J 27, 2342-2354 (2013)) and mass spectrometry. IT-sialidase-treated mucins are characterised in terms of physicochemical properties and used in binding assays (Mackenzie, D. A., Jeffers, F., Parker, M. L., Vibert-Vallet, A., Bongaerts, R. J., Roos, S., Walter, J., Juge, N. Strain-specific diversity of mucus-binding proteins in the adhesion and aggregation properties of Lactobacillus reuteri. Microbiology 156, 3368-78 (2010)), and in growth experiments in the presence of pathogenic strains as described below.

Impact of IT-Sialidase and 2,7-Anhydro-Neu5Ac on Pathogen Growth In Vitro: Ability of the Pathogens to Utilise 2,7-Anhydro-Neu5Ac as a Potential Nutrient

We showed that only IT-sialidase (ITS)-expressing R. gnavus strains (ATCC 29149 and ATCC 35913) can grow anaerobically in presence of mucin (or 3′sialylated lactose), releasing 2,7-anhydro-Neu5Ac (Crost, E. H. et al. (2013)) and demonstrated that ITS was responsible for this enzymatic activity (Tailford et al. (2015)). There was no difference in growth rate between these strains and R. gnavus E1 strain which does not express a sialidase using simple sugars such as glucose as a control. In addition we demonstrated that both R. gnavus strains (ATCC 29149 and ATCC 35913) were able to grow on 2,7-anyhydro-Neu5Ac, as a sole carbon source (Crost et al., 2016). The ability of pathogens to utilise 2,7-anhydroSA as a potential nutrient by growing the cells in presence of 2,7-anhydroSA (or sialic acid as a control) as sole carbon source under anaerobic conditions is tested. Pathogens include Salmonella and C. difficile strains that do not express a sialidase and thus rely on sialic acid released by members of the gut microbiota to proliferate. The ability of these pathogens to utilise 2,7-anhydro-Neu5Ac is tested by growing the cells in vitro with 2,7-anhydro-Neu5Ac synthesised using the enzymatic synthesis as described above, as sole carbon source. Growth is determined spectrophotometrically and analysed using an appropriately developed DMFit program to compare the effect of the carbon source on growth rates. The impact of ITS on the ability of pathogens to grow on mucins purified from human “goblet-like” cells is tested in co-culture experiments with R. gnavus strains (ATCC 29149 expressing ITS and E1 as a negative control) in anaerobic continuous reactors.

A wider range of pathogens, including respiratory pathogens Pseudomonas aeruginosa and Streptococcus pneumonia isolated from human clinical samples are also tested following this method.

Impact of IT-Sialidase-Expressing Gut Bacteria on Pathogens In Vivo

The ability of IT-sialidase (ITS) expressing bacteria to limit pathogen outgrowth in vivo is tested. In our preliminary work, we have shown that R. gnavus ATCC 29149 and ATCC 35913 strains expressing ITS and the E1 strain which does not express a sialidase (as a control) can colonise germ-free mice after gavage with 10⁸ R. gnavus cells then kept in normal cages. R. gnavus, quantified from faecal samples by qPCR as a percent of total 16S rDNA, was still present 28 days post-gavage and no adverse effect was recorded with the ITS-expressing strains. Here, R. gnavus strains (E1 and ATCC 29149 or ATCC 35913) are used in mice (human-like for Neu5Ac) post-antibiotic treatment or Germ Free mice to assess the impact of ITS on i) the modulation of Neu5Ac and 2,7-anhydro-Neu5Ac levels in the gut by HPLC and ii) S. Typhimurium and C. difficile colonisation.

### Example 4

Production of Isotopically-Enriched 2,7-Anhydro-Neu5Ac

As for the non-isotopically-enriched synthesis, recombinant R. gnavus IT-sialidase to be used for the isotopically-enriched enzymatic synthesis of 2,7-anhydro-Neu5Ac is produced in Escherichia coli (as described in Tailford, L. E. et al. (2015)). The experimental approach includes the enzymatic synthesis of isotopically-enriched 2,7-anhydro-Neu5Ac from commercial ¹³C-enriched pyruvate and glucose, via a combination (either one pot or sequential) of isomerase/transaminase/aldolase/transferase/IT-sialidase reactions to obtain ¹³C-labelled 2,7-anhydro-Neu5Ac. This procedure is based on the previous enzymatic synthesis of NeuAc compounds in ¹³C-labelled form (Milton, M. J., Harris, R., Probert, M. A., Field, R. A., Homans, S. W. New conformational constraints in isotopically (13C) enriched oligosaccharides. Glycobiology 8, 147-153 (1998); Probert, M. A., Milton, M. J., Harris, R., Schenkman, S., Brown, J. M., Homans, S. W., Field, R. A. Chemoenzymatic synthesis of GM3, Lewis x and sialyl Lewis x oligosaccharides in ¹³C-enriched form. Tetrahedron Lett 38(33), 5861-64 (1997)). The product of the reaction is analysed by ¹H-NMR and mass spectrometry.

Elucidation of the IT-Sialidase Mediated Metabolism of Sialylated Substrates in R. gnavus Using 2,7-Anhydro-Neu5Ac and ¹³C-2,7-Anhydro-Neu5Ac

Our transcriptomics data showed that the complete cluster dedicated to NeuAc utilisation is induced when R. gnavus ATCC 29149 or ATCC 35913 is grown in presence of sialylated substrates, concomitant with IT-sialidase mediated release of 2,7-anhydro-Neu5Ac, suggesting that 2,7-anhydro-Neu5Ac (and not NeuAc) is transported and metabolised within the cell to support bacterial growth. Transcriptomics analyses of R. gnavus strains grown in presence of this 2,7-anhydro-Neu5Ac (synthesised above) as sole carbon source enables the genes involved in its transport and metabolism inside the cell to be identified. Here ¹³C-labelled probes synthesised according to the procedure above are used to experimentally monitor the fate of 2,7-anhydro-Neu5Ac metabolism into the cell. R. gnavus strains are grown anaerobically in the presence of [¹³C]-2,7-anhydro-Neu5Ac, and the metabolites produced in the cells monitored by multinuclear NMR spectroscopy and mass spectrometry.

Elucidation of the IT-Sialidase Mediated Metabolism of Sialylated Substrates by Gut Bacteria Using ¹³C-2,7-Anhydro-Neu5Ac

A well-established validated in vitro batch model of human colon (Mandalari, G., Nueno-Palop, C., Bisignano, G., Wickham, M., S., Narbad, A. Potential prebiotic properties of almond (Amygdalus communis L.) seeds. Appl Environ Microbiol 74, 4264-4270 (2008)) is seeded with standardised gastrointestinal microbiota from healthy volunteers. After culture stabilisation, ¹³C-2,7-anhydro-Neu5Ac (produced according to the procedure above) is added as isotopically labelled metabolic substrate. Samples are taken from the model at intervals and centrifuged to prepare bacterial pellets and culture supernatants. Metabolite concentrations and isotopic labelling are determined by NMR spectroscopy and mass spectrometry from the supernatants. The total bacterial genomic DNA is extracted from the pellet using established methods and ¹³C-labelled genomic DNA is separated from unlabelled nucleic acids by density-gradient centrifugation.

Profiling Isotopically-Labelled Gut Bacteria by 16S Sequencing

The ¹³C-labelled genomic DNA isolated in the procedure above is used as a template to amplify the V3-V4 region of 16S rDNA genes and then sequenced using the Illumina Miseq platform. Bioinformatics using the standard Quiime software platform reveals the phylogenic structure and identifies the members of the gut microbial community capable of metabolising 2,7-anhydro-Neu5Ac. This allows us to link metabolic activity with microbial. Bioinformatics analyses (on identified bacteria species herein) is carried out to show that 2,7-anhydro-Neu5Ac is preferentially used by IT-sialidase-harbouring bacteria.

Investigation of the Ability of Commensals and Pathogens to Metabolise 2,7-Anhydro-Neu5Ac In Vitro Using 2,7-Anhydro-Neu5Ac and ¹³C-2,7-Anhydro-Neu5Ac

Our bioinformatics analyses revealed the presence of IT-sialidases in other members of the gut microbiota e.g. Blautia hansenii, Ruminococcus torques and Lactobacillus salivarius. Strains are grown in vitro in presence of 3′SL (or mucin), and 6′SL as a control and the supernatant analysed by NMR to monitor the formation of 2,7-anhydro-Neu5Ac (following methods described above). The substrate specificity of these predicted IT-sialidases, following their heterologous production is tested towards a range of sialylated substrates (including mucins and oligosaccharides) by HPAEC and NMR (as previously performed for RgNanH). Growth is monitored in the presence of 2,7-anhydro-Neu5Ac (synthesised above) as sole carbon source to further investigate the ability of these strains to metabolise the released transglycosylation product. ¹³C-2,7-anhydro-Neu5Ac (produced according to the procedure above) is used to monitor Neu5Ac metabolism inside the cell. In contrast to commensals, some enteric pathogens such as Salmonella, Typhimurium or Clostridium difficile strains contain the Nan cluster which allows Neu5Ac metabolism inside the cell but lack the sialidase, thus relying on commensals to acquire this potential nutrient source. Here we test whether such pathogens are able to utilise 2,7-anhydro-Neu5Ac by growing the cells in 2,7-anhydro-Neu5Ac or ¹³C-labelled 2,7-anhydro-Neu5Ac individually or in co-culture with the above-organisms in presence of 3′SL (or mucin) under anaerobic conditions. Other respiratory pathogens, such as Pseudomomonas aeruginosa, are also tested.

### Example 5

The sialic acid metabolic pathway is further validated by functionally characterising the enzymes encoded by R. gnavus Nan cluster. Heterologous expression of the genes from the Nan cluster in Escherichia coli (Tailford et al., 2015) as described above or alternative systems such as Lactococcus lactis are used. The recombinant proteins are subsequently overproduced and purified. Biochemical characterization is performed by incubating an individual enzyme or a combination of enzymes (or transporters) with a particular substrate and analyzing the resulting products by high-performance thin-layer chromatography (HPTLC). Preliminary bioinformatics analysis showed that IT-sialidases are represented in 13% of bacteria possessing a potential Nan cluster, (Tailford et al., 2015). The functional assignment of Nan genes involved in the 2,7-anhydro-Neu5Ac metabolic pathway is used as a platform to refine this analysis. Determination of gut bacteria (commensal and pathogens) that possess a Nan cluster, a sialidase or predicted IT-sialidase, and the combination of sialidase or IT-sialidase together with the Nan cluster will be made to identify and select a range of commensal bacteria that can be used to mop up sialic acid otherwise available to pathogens.

TABLE 1
Kinetic parameters of RgNanH with sialylated substrates

| | | | | k_{cat}/K_{M} | |
|---|---|---|---|---|---|
| Substrate | Vₘₐₓ (μM · min⁻¹) | k_{cat} (min⁻¹) | K_{M} (mM) | (min⁻¹ · mM⁻¹) | Kᵢ (mM) |
| MU-Neu5Ac | 1.37 ± 0.23 | 6.21 × 10³ | 0.59 ± 0.17 | 1.05 × 10⁴ | 2.37 ± 0.65 |
| PNP-Neu5Ac | ND | <152** | >25* | 6.07 × 10³ | ND |
| 3′SL | 5.66 × 10⁻² ± 0.57 × 10⁻² | 25.70 | <<0.1* | >>2.57 × 10²** | 0.76 ± 0.23 |

*estimated value,
**parameter calculated using estimated value,
ND—not detectable. The standard error of the mean is shown.

TABLE 2
Effect of neuraminidase inhibitors on RgNanH activity

| Inhibitor | LogIC₅₀ | IC₅₀ (mM) |
|---|---|---|
| Zanamivir | 1.08 ± 0.07 | 11.89 |
| Neu5Ac2en | 0.15 ± 0.04 | 1.41 |
| Siastatin B | −2.31 ± 0.02 | 4.87 × 10⁻³ |
| Oseltamivir carboxylate | −1.53 ± 0.02 | 2.96 × 10⁻² |

TABLE 3
Specificity of RgNanH and AkmNan 0625 and 1835 against 3′ and 6′ linked sialyl-
oligosaccharides

| | | Activity* | |
|---|---|---|---|
| | | | AkmNan0625 and |
| Substrate (common name) | Systematic name | RgNanH | AkmNan1835 |
| 3′ sialyllactose | Neu5Ac-α-2-3-Gal-β-1-4- | + | + |
| | Glc | | |
| 3′ sialyl-3-fucosyllactose | Neu5Ac-α-2-3-Gal-β1-4- | + | + |
| | [Fuc-α-1-3]-Glc | | |
| 3′-α-sialyl-N- | Neu5Ac-α-2-3-Gal-β-1-4- | + | + |
| acetyllactosamine | GlcNAc | | |
| 3′-sialyl Lewis X | Neu5Ac-α-2-3-Gal-β-1-4- | + | + |
| | [Fuc-a1-3]-GlcNAc | | |
| 3′ sialyl Lewis X methyl | Neu5Ac-α-2-3-Gal-β-1-4- | + | + |
| glycoside | [Fuc-a-1-3]-GlcNAc-β- | | |
| | OMe | | |
| 3′ sialylgalactose | Neu5Ac-α-2-3-Gal | + | + |
| 6′ sialyllactose | Neu5Ac-α-2-6-Gal-β1-4- | − | + |
| | Glc | | |
| 6′-α-sialyl-N- | Neu5Ac-α-2-6-Gal-β-1-4- | − | + |
| acetyllactosamine | GlcNAc | | |
| 6′ sialylgalactose | Neu5Ac-α-2-6-Gal | − | + |

*Activity was defined as disappearance of the substrate peak and appearance of a lower molecular weight peak which could correspond to the desialylated substrate.
Additional abbreviation:
—OMe: —O-Methyl group

TABLE 4
Data collection and refinement statistics*

| | 2,7-anhydro- | | | |
|---|---|---|---|---|
| | Neu5Ac | Neu5Ac2en | oseltamivir carboxylate | Siastatin B |
| PDB | 4X4A | 4X47 | 4X49 | 4X6K |
| identifier | | | | |
| Data | | | | |
| collection | | | | |
| Space group | R3 | R3 | R3 | R3 |
| Cell | | | | |
| dimensions | | | | |
| a, b, c (Å) | 99.26, 99.26, | 101.31, 101.31, | 101.10, 101.10, | 99.11, 99.11, |
| | 130.60 | 131.94 | 131.54 | 131.71 |
| α, β, γ (°) | 90.00, 90.00, | 90.00, 90.00, | 90.00, 90.00, | 90.00, 90.00, |
| | 120.00 | 120.00 | 120.00 | 120.00 |
| Resolution (Å) | 92.54-1.71 | 73.05-2.00 | 72.88-2.01 | 71.91-1.94 |
| | (1.74-1.71) | (2.03-2.00) | (2.04-2.01) | (1.97-1.94) |
| R_{merge} | 0.049 (0.421) | 0.064 (0.490) | 0.068 (0.499) | 0.066 (0.458) |
| I/σI | 41.72 (3.09) | 25.42 (3.17) | 20.32 (2.93) | 25.30 (3.88) |
| Completeness | 92.90 (53.00) | 99.5 (99.7) | 98.3 (97.20) | 98.30 (94.41) |
| (%) | | | | |
| Redundancy | 4.2 (2.4) | 2.9 (2.6) | 2.7 (2.4) | 3.1 (3.0) |
| Refinement | | | | |
| Resolution (Å) | 92.54-1.71 | 73.05-2.00 | 72.88-2.01 | 71.91-1.94 |
| | (1.76-1.71) | (2.05-2.00) | (2.06-2.01) | (2.00-1.94) |
| No. reflections | 51820 | 32166 | 30874 | 32952 |
| R_{work}/R_{free} | 0.761 | 0.788 | 0.751 | 0.790 |
| No. atoms | 4411 | 4080 | 4272 | 4281 |
| Protein | 3836 | 3820 | 3803 | 3798 |
| Ligand/ion | 46 | 25 | 54 | 18 |
| Water | 529 | 235 | 415 | 465 |
| B-factors | | | | |
| Protein | 29.199 | 40.950 | 30.786 | 26.562 |
| Ligand/ion | 37.084 | 39.018 | 34.223 | 20.960 |
| Water | 44.571 | 43.514 | 38.415 | 35.491 |
| R.m.s | | | | |
| deviations | | | | |
| Bond lengths | 0.019 | 0.016 | 0.016 | 0.019 |
| (Å) | | | | |
| Bond angles | 1.891 | 1.787 | 1.802 | 1.923 |
| (°) | | | | |

*Values in parentheses are for the highest-resolution shell. One crystal was used for each structure.

TABLE 5
Presence of IT-sialidase in the MetaHit human samples
Incidence of hits in the HMMER3 search for the sialidase and I-domains in coding sequences
from the MetaHIT study¹⁰. Each sample consists of assembled coding sequences from the gut
metagenome of a single subject designated as negative (N) or positive (Y) for inflammatory
bowel disease (IBD) according to that study.

| | | | | | | positive | | |
|---|---|---|---|---|---|---|---|---|
| | | | | | | samples | | mean hit |
| | | mean | mean | | | (i.e. with | % | sequences |
| subject | no. of | sequences | b.p. per | protein | % hit | ≥1 hit | positive | per |
| group | samples | per sample | sequence | domain | sequences | sequence) | samples | sample |
| IBD = N | 99 | 117,359.99 | 636.90 | Sialidase | 0.0259% | 99 | 100 | 30.44 |
| | | | | I-domain | 0.0001% | 8 | 8 | 0.14 |
| IBD = Y | 25 | 101,351.28 | 637.90 | Sialidase | 0.0268% | 25 | 100 | 27.12 |
| | | | | I-domain | 0.0002% | 5 | 20 | 0.24 |

TABLE 6
Signals of 2,7-anhydro-Neu5Ac and their chemical shifts
Proton chemical shifts for the non-exchangeable protons
of 2,7-anhydro-Neu5Ac in D2O solution.

| | Chemical shift (ppm) | | | | | | |
|---|---|---|---|---|---|---|---|
| | H-3ax | H-3eq | H-4 | H-5 | H-6 | H-7 | 5-Ac |
| H | 2.18 | 2.02 | 4.09 | 3.94 | 4.56 | 4.45 | 2.05 |
| C | 35.44 | 35.44 | 67.85 | 52.16 | 77.01 | 76.63 | 21.93 |

TABLE 7
Occurrence of IT-sialidases and Nan clusters in genome-sequenced bacteria

| | | | | | | Number of genomes | | | | | | |
|---|---|---|---|---|---|---|---|---|---|---|---|---|
| | | | | | | | | | with nan genes | | | |
| | | | | | | | | | “classical” nan | | “B. fragili | |
| | | | | | | | | | genes² | | s-type” | |
| | | | | | | | with | without | | not | nan | without |
| Phylum | Class | Order | Family | Genus | Species | Total¹ | IT-sialidase | IT-sialidase | clustered³ | clustered | genes⁴ | nan genes |
| Firmicutes | Bacilli | Bacillales | Staphylo- | Staphylococcus | S. pseudintermedius | 2 | 2 | 0 | 0 | 2 | 0 | 0 |
| | | | coccaceae | | | | | | | | | |
| | | Lacto- | Lactobacillaceae | Lactobacillus | L. salivarius | 7 | 3 | 4 | 6 | 1 | 0 | 0 |
| | | bacillales | | | | | | | | | | |
| | | | Streptococcaceae | Streptococcus | S. agalactiae | 149 | 137 | 12 | 147 | 2 | 0 | 0 |
| | | | | | S. canis | 1 | 1 | 0 | 1 | 0 | 0 | 0 |
| | | | | | S. equi | 4 | 4 | 0 | 4 | 0 | 0 | 0 |
| | | | | | S. ictaluri | 1 | 1 | 0 | 1 | 0 | 0 | 0 |
| | | | | | S. infantis | 6 | 6 | 0 | 6 | 0 | 0 | 0 |
| | | | | | S. iniae | 2 | 2 | 0 | 2 | 0 | 0 | 0 |
| | | | | | S. intermedius | 6 | 5 | 1 | 6 | 0 | 0 | 0 |
| | | | | | S. mitis | 15 | 13 | 2 | 15 | 0 | 0 | 0 |
| | | | | | S. oralis | 10 | 10 | 0 | 10 | 0 | 0 | 0 |
| | | | | | S. peroris | 1 | 1 | 0 | 1 | 0 | 0 | 0 |
| | | | | | S. pneumoniae | 254 | 254 | 0 | 239 | 15 | 0 | 0 |
| | | | | | S. pseudo-pneumoniae | 3 | 3 | 0 | 3 | 0 | 0 | 0 |
| | | | | | S. sanguinis | 22 | 1 | 21 | 22 | 0 | 0 | 0 |
| | | | | | S. suis | 20 | 2 | 18 | 2 | 18 | 0 | 0 |
| | | | | | S. sp. BS35b | 1 | 1 | 0 | 1 | 0 | 0 | 0 |
| | | | | | S. sp. C300 | 1 | 1 | 0 | 1 | 0 | 0 | 0 |
| | | | | | S. sp. F0441 | 1 | 1 | 0 | 1 | 0 | 0 | 0 |
| | | | | | S. sp. GMD1S | 1 | 1 | 0 | 1 | 0 | 0 | 0 |
| | | | | | S. sp. GMD2S | 1 | 1 | 0 | 0 | 0 | 0 | 1 |
| | | | | | S. sp. GMD4S | 1 | 1 | 0 | 0 | 1 | 0 | 0 |
| | | | | | S. sp. GMD6S | 1 | 1 | 0 | 0 | 1 | 0 | 0 |
| | | | | | S. sp. M143 | 1 | 1 | 0 | 1 | 0 | 0 | 0 |
| | | | | | S. sp. M334 | 1 | 1 | 0 | 1 | 0 | 0 | 0 |
| | | | | | S. sp. SK140 | 1 | 1 | 0 | 1 | 0 | 0 | 0 |
| | | | | | S. sp. SK643 | 1 | 1 | 0 | 1 | 0 | 0 | 0 |
| | | | | | S. sp. oral taxon 058 | 1 | 1 | 0 | 1 | 0 | 0 | 0 |
| | | | | | S. sp. oral taxon 071 | 1 | 1 | 0 | 1 | 0 | 0 | 0 |
| | | | Carnobacteriaceae | Dolosigranulum | D. pigrum | 1 | 1 | 0 | 1 | 0 | 0 | 0 |
| | Erysipelotrichia | Erysipelo- | Erysipelo- | Alloiococcus | A. otitis | 1 | 1 | 0 | 1 | 0 | 0 | 0 |
| | | trichales | trichaceae | Coprobacillus | C. sp. 29_1 | 1 | 1 | 0 | 0 | 1 | 0 | 0 |
| | | | | Erysipelato- | Clostridium spiroforme⁵ | 1 | 1 | 0 | 0 | 1 | 0 | 0 |
| | | | | clostridium | | | | | | | | |
| | Clostridia | Clostridiales | Lachnospiraceae | Blautia | B. hansenii | 1 | 1 | 0 | 1 | 0 | 0 | 0 |
| | | | | | Ruminococcus gnavus^{6,*} | 5 | 4 | 1 | 4 | 1 | 0 | 0 |
| | | | | | Ruminococcus torques^{6,**} | 2 | 1 | 1 | 1 | 0 | 1 | 0 |
| | | | | Tyzzerella | Tyzzerella nexilis⁷ | 1 | 1 | 0 | 0 | 0 | 0 | 1 |
| | | | | unclassified | Lachnospiraceae bacterium | 1 | 1 | 0 | 0 | 0 | 1 | 0 |
| | | | | Lachnospiraceae | 1_1_57FAA | | | | | | | |
| | | | | | Lachnospiraceae bacterium | 1 | 1 | 0 | 0 | 0 | 1 | 0 |
| | | | | | 3_1_46FAA | | | | | | | |
| | | | | | Lachnospiraceae bacterium | 1 | 1 | 0 | 0 | 0 | 1 | 0 |
| | | | | | 8_1_57FAA | | | | | | | |
| | | | Clostridiaceae | Clostridium | C. sp. 7_2_43FAA | 1 | 1 | 0 | 1 | 0 | 0 | 0 |
| | | | | | C. celatum | 1 | 1 | 0 | 0 | 1 | 0 | 0 |
| | | | | | C. perfringens | 10 | 10 | 0 | 10 | 0 | 0 | 0 |
| Proteobacteria | Gamma- | Pasteurellales | Pasteurellaceae | Pasteurella | P. multocida | 11 | 8 | 3 | 9 | 2 | 0 | 0 |
| | proteobacteria | | | | | | | | | | | |

¹Number of genomes with predicted protein-coding genes present in the NCBI Genomes database; a few strains of S. infantis, S. oralis and S. pneumoniae are represented by more than one genome.
²NanA, NanK, NanE(1) (R. gnavus nomenclature)
³the 3 nan genes are within a maximum of 15 consecutive genes.
⁴NanL, NanE(2), RokA (B. fragilis nomenclature)
⁵Clostridium spiroforme has been reclassified (Yutin and Galperin, 2013)
⁶R. gnavus and R. torques belong to a group of Ruminococcus spp. that have been reclassified in the genus Blautia, but have so far retained the Ruminococcus genus name (Liu et al, 2008).
⁷Formerly Clostridium nexile (Yutin and Galperin, 2013)
*One of the 5 genomes analyzed belongs to the E1 strain and is not publicly available.
**One R. torques strain, which lacks the IT-sialidase, has the traditional NanA/K/E(1) cluster instead of the B. fragilis components.

SEQUENCES

RgNanH amino acid sequence including 25 amino acid signal
sequence (SEQ ID NO: 1)
MNKYKKIVSIAATAVMCVAPMSVYAQEAQTDVIEAVAEKKQDTESSSVPVLQKEGIEISE
GTGYDLSKEPGAATVKALEQGTIVISYKTTSENAIQSLLSVGNGTKGNQDRHFHLYITNA
GGVGMELRNIDGEFKYTLDCPAAVRGSYKGERVSNTVALKADKENKQYKLFANGELIATL
DQEAFKFISDITGVDNVMLGGTMRQGTVAYPFGGSIERMQVYRDVLSDDELIAVTGKTIY
AENIFYAGDATKSNYFRIPSLLALDSGTVIAAADARYGGTHDAKSKINTAFAKSTDGGKT
WGQPTLPLKFDDYVAKNIDWPRDSVGKNVQIQGSASYIDPVLLEDKETHRVFLFADMMPA
GIGSSNASVGSGFKEVDGKKYLKLHWKDDAAGTYDYSVRENGTIYNDTTNSATEYSVDGE
YNLYKNGNAMLCKQYDYNFEGTKLLETQTDTDVNMNVFYKDADFKVFPTTYLAMKYSDDE
GETWSDLQIVSTFKPEESKFLVLGPGVGKQIANGEHAGRLIVPLYSKSSAELGFMYSDDH
GNNWTYVEADQNTGGATAEAQIVEMPDGSLKTYLRTGSGYIAQVMSTDGGETWSERVPLT
EIATTGYGTQLSVINYSQPVDGKPAILLSAPNATNGRKNGKIWIGLISETGNSGKDKYSV
DWKYCYSVDTPQMGYSYSCLTELPDGEIGLLYEKYDSWSRNELHLKNILKYERFNIDELK
VQP
RgNanH nucleic acid sequence (SEQ ID NO: 2)
ATGAATAAGTATAAAAAAATTGTTTCTATAGCAGCAACTGCAGTGATGTGTGTTGCACCA
ATGAGTGTATATGCCCAAGAGGCCCAGACAGATGTGATTGAGGCTGTAGCAGAGAAGAAA
CAAGATACAGAATCTTCTTCGGTACCAGTGTTGCAAAAGGAAGGAATCGAAATCTCGGAA
GGTACAGGATATGATTTGAGTAAAGAACCTGGGGCAGCAACAGTAAAAGCATTGGAACAG
GGAACTATCGTTATCTCCTATAAAACAACCAGTGAAAATGCGATTCAATCGTTATTGAGT
GTGGGAAATGGTACAAAAGGAAATCAGGATAGACATTTCCACTTATATATCACAAATGCA
GGCGGCGTAGGTATGGAATTGAGAAATACAGATGGCGAGTTTAAATATACGCTGGATTGC
CCGGCTGCTGTGCGTGGTAGTTATAAGGGGGAAAGAGTATCCAACACAGTTGCATTAAAA
GCGGACAAAGAAAATAAACAGTACAAGCTATTTGCAAATGGCGAATTAATAGCAACGCTG
GATCAGGAGGCATTTAAGTTTATCAGTGATATTACAGGAGTAGACAATGTAATGCTGGGC
GGTACCATGCGTCAGGGAACCGTTGCCTATCCATTTGGAGGTTCCATAGAGAGAATGCAG
GTATATCGGGATGTACTTTCTGATGATGAACTTATTGCTGTGACAGGAAAGACTATATAT
GCAGAGAATATCTTTTATGCAGGAGATGCTACAAAATCCAACTATTTCCGAATCCCATCC
CTTTTGGCGTTGGATTCTGGTACAGTGATTGCAGCAGCAGATGCAAGATATGGTGGCACC
CATGATGCAAAAAGTAAGATCAATACAGCATTTGCGAAAAGTACAGATGGTGGAAAAACG
TGGGGACAGCCGACATTGCCATTGAAGTTTGACGATTATGTAGCAAAGAACATAGACTGG
CCACGGGATTCGGTGGGCAAGAATGTGCAGATACAAGGGAGTGCTTCCTACATAGATCCG
GTACTTTTAGAGGATAAGGAAACACATCGGGTATTTCTTTTTGCTGATATGATGCCGGCA
GGAATTGGAAGTTCCAACGCTTCCGTAGGTTCTGGATTTAAAGAGGTTGATGGGAAGAAA
TATTTGAAATTGCACTGGAAGGATGATGCGGCAGGAACGTATGATTACTCTGTAAGAGAA
AATGGAACCATTTATAACGATACTACAAATTCAGCAACTGAATATAGTGTTGATGGTGAA
TATAATCTGTACAAAAATGGAAATGCCATGCTATGTAAACAGTATGATTACAATTTTGAA
GGTACAAAGTTGTTGGAAACACAGACGGACACGGACGTAAATATGAATGTTTTTTATAAA
GATGCAGATTTTAAAGTATTCCCAACAACTTATTTGGCAATGAAATACTCAGATGATGAG
GGAGAAACCTGGTCTGATTTACAGATTGTAAGTACCTTTAAGCCAGAGGAATCAAAATTT
CTCGTATTGGGACCAGGTGTAGGCAAACAGATAGCAAATGGAGAACATGCCGGAAGATTG
ATTGTTCCATTGTATTCGAAATCATCTGCAGAACTGGGATTTATGTATAGTGATGACCAT
GGAAATAACTGGACATACGTAGAAGCAGACCAGAATACAGGTGGGGCAACCGCAGAAGCA
CAGATCGTAGAAATGCCGGATGGTTCCTTGAAAACTTATCTGCGTACAGGATCAGGATAT
ATTGCACAAGTGATGAGTACAGATGGAGGAGAAACCTGGAGTGAGCGAGTTCCACTAACA
GAAATTGCAACAACAGGGTATGGAACGCAGTTGTCAGTGATTAATTATTCACAGCCTGTT
GATGGAAAGCCTGCTATCTTACTATCAGCACCAAATGCGACCAATGGCAGAAAAAATGGA
AAGATATGGATTGGTTTGATCAGCGAAACAGGAAATTCCGGAAAAGATAAGTATTCTGTA
GATTGGAAATATTGCTATTCTGTGGATACCCCTCAAATGGGCTACTCTTATTCCTGTCTG
ACAGAGCTTCCGGATGGCGAGATCGGGCTTTTATATGAAAAATATGATTCCTGGTCAAGA
AATGAATTGCATCTGAAAAATATATTGAAATATGAGAGGTTTAATATTGATGAACTGAAA
GTTCAACCATAA
RgNanH amino acid sequence excluding 25 amino acid signal
sequence (SEQ ID NO: 3)
QEAQTDVIEAVAEKKQDTESSSVPVLQKEGIEISEGTGYDLSKEPGAATVKALEQGTIVISYKT
TSENAIQSLLSVGNGTKGNQDRHFHLYITNAGGVGMELRNTDGEFKYTLDCPAAVRGSYKGERV
SNTVALKADKENKQYKLFANGELIATLDQEAFKFISDITGVDNVMLGGTMRQGTVAYPFGGSIE
RMQVYRDVLSDDELIAVTGKTIYAENIFYAGDATKSNYFRIPSLLALDSGTVIAAADARYGGTH
DAKSKINTAFAKSTDGGKTWGQPTLPLKFDDYVAKNIDWPRDSVGKNVQIQGSASYIDPVLLED
KETHRVFLFADMMPAGIGSSNASVGSGFKEVDGKKYLKLHWKDDAAGTYDYSVRENGTIYNDTT
NSATEYSVDGEYNLYKNGNAMLCKQYDYNFEGTKLLETQTDTDVNMNVFYKDADFKVFPTTYLA
MKYSDDEGETWSDLQIVSTFKPEESKFLVLGPGVGKQIANGEHAGRLIVPLYSKSSAELGPMYS
DDHGNNWTYVEADQNTGGATAEAQIVEMPDGSLKTYLRTGSGYIAQVMSTDGGETWSERVPLTE
LATTGYGTQLSVINYSQPVDGKPAILLSAPNATNGRKNGKIWIGLISEIGNSGKDKYSVDWKYC
YSVDTPQMGYSYSCLTELPDGEIGLLYEKYDSWSRNELHLKNILKYERFNIDELKVQP
RgNanH Signal peptide (SEQ ID NO: 4)
MNKYKKIVSIAATAVMCVAPMSVYA
RgNan CBM40 (SEQ ID NO: 5)
QEAQTDVIEAVAEKKQDTESSSVPVLQKEGIEISEGTGYDLSKEPGAATVKALEQGTIVISYKT
TSENAIQSLLSVGNGTKGNQDRHFHLYITNAGGVGMELRNTDGEFKYTLDCPAAVRGSYKGERV
SNTVALKADKENKQYKLFANGELIATLDQEAFKFISDITGVDNVMLGGTMRQGTVAYPFGGSIE
RMQVYRDVLSDDELIAVTGKTIY
RgNan GH33 (SEQ ID NO: 6)
AENIFYAGDATKSNYFRIPSLLALDSGTVIAAADARYGGTHDAKSKINTAFAKSTDGGKTWGQP
TLPLKFDDYVAKNIDWPRDSVGKNVQIQGSASYIDPVLLEDKETHRVFLFADMMPAGIGSSNAS
VGSGFKEVDGKKYLKLHWKDDAAGTYDYSVRENGTIYNDTTNSATEYSVDGEYNLYKNGNAMLC
KQYDYNFEGTKLLETQTDTDVNMNVFYKDADFKVFPTTYLAMKYSDDEGETWSDLQIVSTFKPE
ESKFLVLGPGVGKQIANGEHAGRLIVPLYSKSSAELGPMYSDDHGNNWTYVEADQNTGGATAEA
QIVEMPDGSLKTYLRTGSGYIAQVMSTDGGETWSERVPLTEIATTGYGTQLSVINYSQPVDGKP
AILLSAPNATNGRKNGKIWIGLISETGNSGKDKYSVDWKYCYSVDTPQMGYSYSCLTELPDGEI
GLLYEKYDSWSRNELHLKNILKYERFNIDELKVQP

1. A method for production of a 2,7-anhydro sialic acid derivative comprising incubating a substrate with an IT-sialidase, or an enzymatic fragment thereof, under conditions for enzymatic activity.

2. A method as claimed in claim 1 wherein the IT-sialidase is an IT-sialidase having an amino acid sequence with 80% identity to the amino acid sequence set out in SEQ ID NO:1.

3. A method as claimed in claim 2 wherein the IT-sialidase is RgNanH, having the amino acid sequence set out in SEQ ID NO:1.

4. A method as claimed in claim 1 wherein the method uses an enzymatic fragment of an IT-sialidase.

5. A method as claimed in claim 4 wherein the enzymatic fragment is RgGH33 having the amino acid sequence set out in SEQ ID NO:6.

6. A method for production of a 2,7-anhydro sialic acid derivative as claimed in claim 1 wherein the 2,7-anhydro sialic acid derivative is 2,7-anhydro-Neu5Ac, 2,7-anhydro-Neu5Gc or 2,7-anhydro-KDN.

7. A method as claimed in claim 1 wherein the substrate is an alpha2,3 linked sialic acid-containing substrate, such as fetuin, asialofetuin, mucin, 4MU-Neu5Ac, Neu5Acα2-3Lac, N-glycolylneuraminic acid (Neu5Gc), Neu5Gcα2-3Lac, Kdn-alpha2-3Lac or 2-keto-3-deoxy-d-glycero-d-galacto-nononic acid (KDN).

8. A method as claimed in claim 1 wherein the incubation is conducted in a membrane enclosed environment.

9. A method as claimed in claim 1 wherein the incubation of an alpha2,3 linked sialic acid-containing substrate with an IT-sialidase is followed by incubation with a sialic acid modifying enzyme, such as a lyase or sialic acid aldolase.

10. An IT-sialidase having an amino acid sequence with 80% identity to the amino acid sequence set out in SEQ ID NO 1.

11. An IT-sialidase as claimed in claim 10 wherein said IT-sialidase has an enzymatic activity of producing 2,7-anhydro-Neu5Ac from alpha2-3-linked sialic acid substrates.

12. An IT-sialidase as claimed in claim 10, wherein said IT-sialidase is derived from bacteria of the gut microbiota.

13. An IT-sialidase as claimed in claim 12 wherein said IT-sialidase is derived from R. gnavus ATCC 29149.

14. An IT-sialidase as claimed in claim 10 having the amino acid sequence set out in SEQ ID NO: 1.

15. An isolated nucleic acid molecule encoding an IT-sialidase as claimed in claim 10.

16. A vector comprising a nucleic acid as claimed in claim 15.

17. A host cell comprising a nucleic acid as claimed in claim 15.

18. A host cell as claimed in claim 17 wherein said cell is a microorganism.

19. An expression system for expressing IT-sialidase comprising a host cell as claimed in claim 17.

20. A method for the production of an IT-sialidase as claimed in claim 10 wherein said method comprises culturing a host cell under conditions suitable for expression of said nucleic acid molecule or vector to produce an IT-sialidase.

21. A pharmaceutical composition comprising an IT-sialidase as claimed in claim 10 and one or more pharmaceutically acceptable carriers or excipients.

22. An IT-sialidase as claimed of claim 10 for use in therapy.

23. A pharmaceutical composition comprising an IT-sialidase-expressing microorganism and one or more pharmaceutically acceptable carriers or excipients.

24.-52. (canceled)
